(19) United States
(12) Patent Application Publication
(10) Pub. No.: US 2021/0260378 A1
(43) Pub. Date: Aug. 26, 2021

(54) SLEEP-LINKED ADJUSTMENT METHODS FOR PROSTHESES

(71) Applicant: Cochlear Limited, Macquarie University, NSW (AU)

(72) Inventors: Wendy POTTS, Macquarie University (AU); Naomi CROGHAN, Macquarie University (AU); Christopher Joseph LONG, Macquarie University (AU); Zachary Mark SMITH, Macquarie University (AU)

(21) Appl. No.: 17/261,650

(22) PCT Filed: Aug. 30, 2019
(86) PCT No.: PCT/IB2019/057345

§ 371 Date: Jan. 20, 2021

Related U.S. Application Data
(60) Provisional application No. 62/725,592, filed on Aug. 31, 2018.

(51) Int. Cl. A61N 1/36; H04R 25/00

(52) U.S. Cl. CPC A61N 1/36039; H04R 2225/55; H04R 25/554; H04R 25/606

(57) ABSTRACT

A method, including providing stimulation to a recipient of a prosthesis, such as a hearing prosthesis, such as an implantable prosthesis, such as a cochlear implant, wherein the stimulation is provided at temporal locations associated with sleep of the recipient, and the stimulation is at least one of part of a measurement method or an auditory training method.

### CROSS-REFERENCE TO RELATED APPLICATIONS

This application claims priority to U.S. Provisional Application No. 62/725,592, entitled SLEEP-LINKED ADJUSTMENT METHODS FOR PROSTHESES, filed on Aug. 31, 2018, naming Wendy POTTS of Centennial, Colo. as an inventor, the entire contents of that application being incorporated herein by reference in its entirety.

### BACKGROUND

Hearing loss, which may be due to many different causes, is generally of two types: conductive and sensorineural. Sensorineural hearing loss is due to the absence or destruction of the hair cells in the cochlea that transduce sound signals into nerve impulses. Various hearing prostheses are commercially available to provide individuals suffering from sensorineural hearing loss with the ability to perceive sound.

Conductive hearing loss occurs when the normal mechanical pathways that provide sound to hair cells in the cochlea are impeded, for example, by damage to the ossicular chain or the ear canal. Individuals suffering from conductive hearing loss may retain some form of residual hearing because the hair cells in the cochlea may remain undamaged.

Individuals suffering from sensorineural hearing loss typically receive an acoustic hearing aid. Conventional hearing aids rely on principles of air conduction to transmit acoustic signals to the cochlea. In particular, a hearing aid typically uses an arrangement positioned in the recipient's ear canal or on the outer ear to amplify a sound received by the outer ear of the recipient. This amplified sound reaches the cochlea causing motion of the perilymph and stimulation of the hair cells in the cochlear, which stimulate the auditory nerve. Cases of conductive hearing loss typically are treated by means of bone conduction hearing aids. In contrast to conventional hearing aids, these devices use a mechanical actuator that is coupled to the skull bone to apply the amplified sound.

In contrast to hearing aids, which rely primarily on the principles of air conduction, certain types of hearing prostheses, commonly referred to as cochlear implants, convert a received sound into electrical stimulation. The electrical stimulation is applied to the cochlea, which results in the perception of the received sound.

It is noted that in at least some instances, there is utilitarian value to fitting a hearing prosthesis to a particular recipient. In some examples of some fitting regimes, there are methods which entail a clinician or some other professional presenting sounds to a recipient of the hearing prosthesis such that the hearing prosthesis evokes a hearing percept.

### SUMMARY

In accordance with an exemplary embodiment, there is a method, comprising providing stimulation to a recipient of a hearing prosthesis, wherein the stimulation is provided at temporal locations associated with sleep of the recipient, and the stimulation is at least one of part of a measurement method or an auditory training method.

In an exemplary embodiment, there is a method, comprising receiving input indicative of measurements executed using a hearing prosthesis while the recipient thereof is sleeping, analyzing the received input and at least one of adjusting a setting of the hearing prosthesis or loading a new setting of the hearing prosthesis based on the analysis.

In an exemplary embodiment, there is a non-transitory computer readable medium having recorded thereon, a computer program for executing a method, the program including code for determining a feature indicative of a sleep state of a recipient of a hearing prosthesis, and code for implementing measurements of the recipient based on the determination of the sleep state.

Also, in another exemplary embodiment, there is a system, comprising a first sub-system configured to obtain data indicative of a sleep state of a recipient of a sensory prosthesis and a second sub-system configured to execute measurements of the recipient.

### BRIEF DESCRIPTION OF THE DRAWINGS

Embodiments are described below with reference to the attached drawings, in which:

FIG. 1 is a perspective view of an exemplary hearing prosthesis in which at least some of the teachings detailed herein are applicable;

FIG. 2 presents an exemplary electrode array according to an exemplary embodiment;

FIG. 3 presents an exemplary device in use according to an exemplary arrangement;

FIG. 4 presents exemplary flowchart for an exemplary method;

FIG. 5 presents another exemplary flowchart for another exemplary method;

FIG. 6 presents a combined exemplary flowchart as well as a functional diagram according to an exemplary embodiment;

FIGS. 7 and 8 present black box diagrams according to exemplary embodiments;

FIGS. 9-16 present exemplary flowcharts for exemplary algorithms according to exemplary embodiments; and

FIG. 17 presents an exemplary black box diagram for an exemplary system.

### DETAILED DESCRIPTION

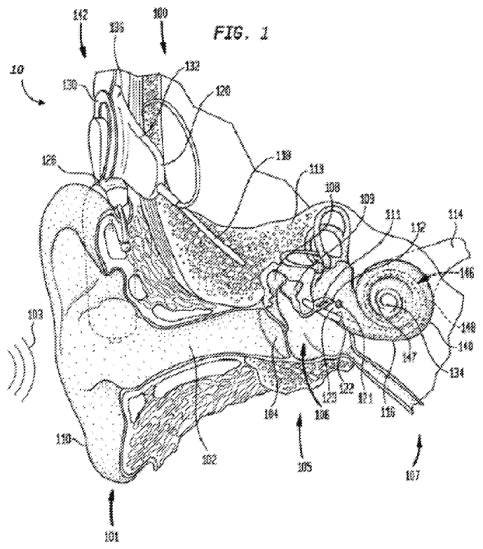

FIG. 1 is a perspective view of a cochlear implant, referred to as cochlear implant 100, implanted in a recipient, to which some embodiments detailed herein and/or variations thereof are applicable. The cochlear implant 100 is part of a system 10 that can include external components, in some embodiments, as will be detailed below. It is noted that the teachings detailed herein are applicable, in at least some embodiments, to partially implantable and/or totally implantable cochlear implants (i.e., with regard to the latter, such as those having an implanted microphone). It is further noted that the teachings detailed herein are also applicable to other stimulating devices that utilize an electrical current beyond cochlear implants (e.g., auditory brain stimulators, pacemakers, retinal implants, etc.).

Additionally, it is noted that the teachings detailed herein are also applicable to other types of hearing prostheses, such as by way of example only and not by way of limitation, bone conduction devices (percutaneous, active transcutaneous and/or passive transcutaneous), direct acoustic cochlear stimulators, middle ear implants, and conventional hearing aids, etc. Indeed, it is noted that the teachings detailed herein are also applicable to so-called multi-mode devices. In an exemplary embodiment, these multi-mode devices apply both electrical stimulation and acoustic stimulation to the recipient. In an exemplary embodiment, these multi-mode devices evoke a hearing percept via electrical hearing and bone conduction hearing. Accordingly, any disclosure herein with regard to one of these types of hearing prostheses corresponds to a disclosure of another of these types of hearing prostheses or any medical device for that matter, unless otherwise specified, or unless the disclosure thereof is incompatible with a given device based on the current state of technology. Thus, the teachings detailed herein are applicable, in at least some embodiments, to partially implantable and/or totally implantable medical devices that provide a wide range of therapeutic benefits to recipients, patients, or other users, including hearing implants having an implanted microphone, auditory brain stimulators, pacemakers, visual prostheses (e.g., bionic eyes), sensors, drug delivery systems, defibrillators, functional electrical stimulation devices, catheters, etc.

In view of the above, it is to be understood that at least some embodiments detailed herein and/or variations thereof are directed towards a body-worn sensory supplement medical device (e.g., the hearing prosthesis of FIG. 1, which supplements the hearing sense, even in instances when there are no natural hearing capabilities, for example, due to degeneration of previous natural hearing capability or to the lack of any natural hearing capability, for example, from birth). It is noted that at least some exemplary embodiments of some sensory supplement medical devices are directed towards devices such as conventional hearing aids, which supplement the hearing sense in instances where some natural hearing capabilities have been retained, and visual prostheses (both those that are applicable to recipients having some natural vision capabilities and to recipients having no natural vision capabilities). Accordingly, the teachings detailed herein are applicable to any type of sensory supplement medical device to which the teachings detailed herein are enabled for use therein in a utilitarian manner. In this regard, the phrase sensory supplement medical device refers to any device that functions to provide sensation to a recipient irrespective of whether the applicable natural sense is only partially impaired or completely impaired, or indeed never existed.

Returning to FIG. 1, the recipient has an outer ear 101, a middle ear 105, and an inner ear 107. Components of outer ear 101, middle ear 105, and inner ear 107 are described below, followed by a description of cochlear implant 100.

In a fully functional ear, outer ear 101 comprises an auricle 110 and an ear canal 102. An acoustic pressure or sound wave 103 is collected by auricle 110 and channeled into and through ear canal 102. Disposed across the distal end of ear channel 102 is a tympanic membrane 104 which vibrates in response to sound wave 103. This vibration is coupled to oval window or fenestra ovalis 112 through three bones of middle ear 105, collectively referred to as the ossicles 106 and comprising the malleus 108, the incus 109, and the stapes 111. Bones 108, 109, and 111 of middle ear 105 serve to filter and amplify sound wave 103, causing oval window 112 to articulate, or vibrate in response to vibration of tympanic membrane 104. This vibration sets up waves of fluid motion of the perilymph within cochlea 140. Such fluid motion, in turn, activates tiny hair cells (not shown) inside of cochlea 140. Activation of the hair cells causes appropriate nerve impulses to be generated and transferred through the spiral ganglion cells (not shown) and auditory nerve 114 to the brain (also not shown) where they are perceived as sound.

As shown, cochlear implant 100 comprises one or more components which are temporarily or permanently implanted in the recipient. The implantable component of the cochlear implant 100 is shown in FIG. 1 with an external device 142, that is part of cochlear implant system 10 (along with the implantable component of the cochlear implant 100), which, as described below, is configured to provide power to the cochlear implant, where the implanted cochlear implant includes a battery or other energy storage device (e.g., capacitor) that is charged (e.g., recharged) by the power provided from the external device 142. It is briefly noted that sometimes herein the entire system 10 is simply referred to as the cochlear implant, while the implantable component is sometimes referred to as the cochlear implant. Any reference to one corresponds to a reference to the other unless otherwise noted.

In the illustrative arrangement of FIG. 1, external device 142 can comprise a power source (not shown) disposed in a Behind-The-Ear (BTE) unit 126. External device 142 also includes components of a transcutaneous energy transfer link, referred to as an external energy transfer assembly. The transcutaneous energy transfer link is used to transfer power and/or data to cochlear implant 100. Various types of energy transfer, such as infrared (IR), electromagnetic, capacitive and inductive transfer, may be used to transfer the power and/or data from external device 142 to cochlear implant 100. In the illustrative embodiments of FIG. 1, the external energy transfer assembly comprises an external coil 130 that forms part of an inductive radio frequency (RF) communication link. External coil 130 is typically a wire antenna coil comprised of multiple turns of electrically insulated single-strand or multi-strand platinum or gold wire. External device 142 also includes a magnet (not shown) positioned within the turns of wire of external coil 130. It should be appreciated that the external device shown in FIG. 1 is merely illustrative, and other external devices may be used with embodiments of the present invention.

Cochlear implant 100 comprises an internal energy transfer assembly 132 which can be positioned in a recess of the temporal bone adjacent auricle 110 of the recipient. As detailed below, internal energy transfer assembly 132 is a component of the transcutaneous energy transfer link and receives power and/or data from external device 142. In the illustrative embodiment, the energy transfer link comprises an inductive RF link, and internal energy transfer assembly 132 comprises a primary internal coil 136. Internal coil 136 is typically a wire antenna coil comprised of multiple turns of electrically insulated single-strand/or multi-strand platinum or gold wire.

Cochlear implant 100 further comprises a main implantable component 120 and an elongate electrode assembly 118. In some embodiments, internal energy transfer assembly 132 and main implantable component 120 are hermetically sealed within a biocompatible housing. In some embodiments, main implantable component 120 includes an implantable microphone assembly (not shown) and a sound processing unit (not shown) to convert the sound signals received by the implantable microphone in internal energy transfer assembly 132 to data signals. That said, in some alternative embodiments, the implantable microphone assembly can be located in a separate implantable component (e.g., that has its own housing assembly, etc.) that is in signal communication with the main implantable component 120 (e.g., via leads or the like between the separate implantable component and the main implantable component 120). In at least some embodiments, the teachings detailed herein and/or variations thereof can be utilized with any type of implantable microphone arrangement.

Main implantable component 120 further includes a stimulator unit (also not shown) which generates electrical stimulation signals based on the data signals. The electrical stimulation signals are delivered to the recipient via elongate electrode assembly 118.

Elongate electrode assembly 118 has a proximal end connected to main implantable component 120, and a distal end implanted in cochlea 140. Electrode assembly 118 extends from main implantable component 120 to cochlea 140 through mastoid bone 119. In some embodiments, electrode assembly 118 may be implanted at least in basal region 116, and sometimes further. For example, electrode assembly 118 may extend towards apical end of cochlea 140, referred to as cochlea apex 134. In certain circumstances, electrode assembly 118 may be inserted into cochlea 140 via a cochleostomy 122. In other circumstances, a cochleostomy may be formed through round window 121, oval window 112, the promontory 123 or through an apical turn 147 of cochlea 140.

Electrode assembly 118 comprises a longitudinally aligned and distally extending array 146 of electrodes 148, disposed along a length thereof. As noted, a stimulator unit generates stimulation signals which are applied by electrodes 148 to cochlea 140, thereby stimulating auditory nerve 114.

Because the cochlea is tonotopically mapped (i.e., spatial locations that are responsive to stimulus signals in a particular frequency range are identified), frequencies may be allocated to one or more electrodes of the electrode assembly to generate an electric field in positions in the cochlea that are close to the region that would naturally be stimulated in normal hearing. This enables the prosthetic hearing implant to bypass the hair cells in the cochlea to directly deliver electrical stimulation to auditory nerve fibers, thereby allowing the brain to perceive hearing sensations resembling natural hearing sensations. In achieving this, processing channels of the sound processing unit of the BTE 126 (i.e., specific frequency bands with their associated signal processing paths), are mapped to a set of one or more electrodes to stimulate a desired nerve fiber or nerve region of the cochlea. Such sets of one or more electrodes for use in stimulation are referred to herein as “electrode channels” or “stimulation channels.” In at least some exemplary embodiments, each channel has a “base” electrode corresponding to the electrode of the electrode array that is proximate the tonotopically mapped cochlea for a given frequency or frequency range.

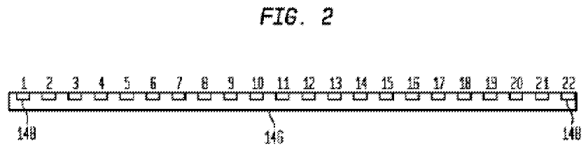

FIG. 2 illustrates a more detailed view, albeit functionally, of an exemplary electrode array 146 comprising a plurality of electrodes 148 labeled 1-22, in accordance with an embodiment. In an exemplary embodiment, each electrode 148 is an electrode that corresponds to a specific frequency band channel of the cochlear implant 100, where electrode 22 corresponds to the lowest frequency band (channel), and electrode 1 corresponds to the highest frequency band (channel), as will be discussed in greater detail below. Briefly, it is noted that during stimulation by the electrodes to evoke a hearing percept, one or more electrodes 148 is activated at a given electrode stimulation level (e.g., current level). This electrode stimulation level is pre-set during a fitting process. For example, in at least some instances, an audiologist adjusts stimulation channel electrode current levels of the cochlear implant 100 based on empirical data. More specifically, in at least some scenarios, stimulation channel electrode current levels are adjusted by an audiologist based on threshold and comfort levels. Then, in at least some scenarios, the cochlear implant 100 is configured such that respective stimulation channels of the cochlear implant 100 have those respective current levels. This can be done, for example, by programming the cochlear implant 100 or by any other process that sets the channels of the cochlear implant 100 to have the pertinent electrical stimulation levels. Any arrangement of the cochlear implant 100 and/or other equipment/devices that will enable the teachings detailed herein and/or variations thereof to be practiced can be used in at least some embodiments.

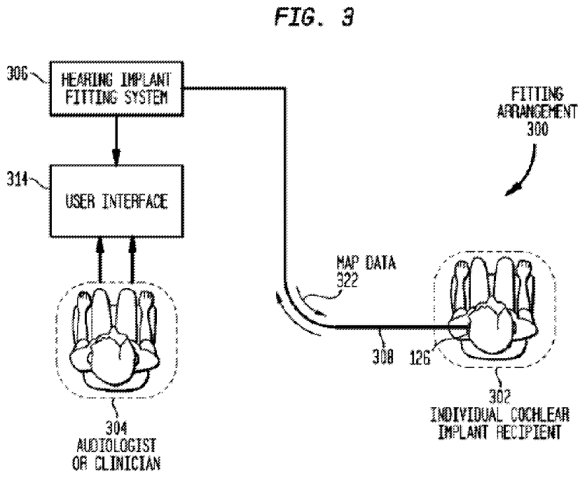

FIG. 3 is a schematic diagram illustrating one exemplary arrangement 300 in which a hearing implant fitting system 306 may be used to fit a cochlear implant, in accordance with an embodiment. As shown in FIG. 3, an audiologist or clinician 304 may use a hearing implant fitting system 306 (“fitting system” herein) comprising interactive software and computer hardware to create individualized recipient map data 322 that are digitally stored on system 306, and ultimately downloaded to the memory of the sound processing unit 126 for recipient 302. System 306 may be programmed and/or implement software programmed to carry out one or more of the functions of mapping, neural response measuring, acoustic stimulating, and recording of neural response measurements and other stimuli.

In the embodiment illustrated in FIG. 3, sound processing unit 126 of cochlear implant 100 may be connected directly to fitting system 306 to establish a data communication link 308 between the sound processing unit 126 and fitting system 306. System 306 is thereafter bi-directionally coupled by a data communication link 308 with sound processing unit 126. It should be appreciated that although sound processing unit 126 and fitting system 306 are connected via a cable in FIG. 3, any communications link now or later developed may be utilized to communicably couple the implant and fitting system.

Some exemplary embodiments will now be described. It is noted that in an exemplary embodiment, the system of FIG. 3 can be utilized in at least some of the teachings detailed below or otherwise to implement at least some of the teachings detailed below, while in other embodiments, the system is not necessarily utilized. It is noted that the following is but exemplary, and that alternative methods can be practiced utilizing other devices other than the fitting system 306 and/or alternative methods can be practiced to fit a prosthesis that is different than cochlear implant 10.

Briefly, at least some teachings detailed herein and/or variations thereof are applicable to the development of a map for a cochlear implant user. As will be detailed herein, the teachings detailed herein and/or variations thereof can be applicable to other types of hearing prostheses other than a cochlear implant. Still further, the teachings detailed herein and/or variations thereof can be applicable in at least some embodiments to hybrid devices and bimodal devices that utilize the cochlear implant along with another type of hearing device (e.g., a traditional hearing aid).

More specifically, in at least some exemplary embodiments, there is an algorithm that enables the development, including the automatic development, of a new electrical output map such that the cochlear implant operates differently than that which was previously the case with

[symbol]

In at least some exemplary embodiments, the cochlear implant includes one or more MAPs stored therein. MAPs are programs that are used in combination with other components to control the input to the electrodes on the array that are implanted into the cochlea. In an exemplary embodiment, the cochlear implant is mapped. In an exemplary embodiment, the cochlear implant processor is connected to the audiologist's computer for MAPping. Using a series of “beeps,” and measuring the CI user's response, the audiologist sets T- and C-levels for each electrode. The audiologist might also adjust the stimulation rate or programming strategy used for the MAP—these refer to the various computer algorithms and programs used to translate acoustic sound (what people with typical hearing perceive) into the correct combination of electrode stimulations to give the cochlear implant user that same sensation of sound. The finalized map is loaded into the cochlear implant or otherwise stored therein, and the recipient utilizes the cochlear implant with that map to evoke a hearing percept based on sound captured by the implant or otherwise provided to the implant via an audio signal.

The map can be adjusted or otherwise replaced during the temporal period extending after the initial mapping. By way of example only and not by way of limitation, in an exemplary scenario, the recipient can experience a fitting session with an audiologist where the cochlear implant is fitted to the recipient, and the map associated or otherwise that results from that fitting is stored into the cochlear implant. The recipient then goes on with life for a couple weeks or a couple of months and then returns to the audiologist to have the map adjusted or the map replaced with a new map. In some instances, the audiologist subjects the recipient to a series of tests or otherwise a series of measurements are taken of the recipient, and the data from those measurements is utilized to adjust the map or otherwise develop a new map. This adjusted map or otherwise developed new map is then loaded or otherwise stored in the cochlear implant, and the recipient than goes on with life until the next mapping session, etc. It is noted that any reference to adjusting the map herein corresponds to a disclosure of developing a new map, and vice versa, unless otherwise noted. It is also noted that the term “settings” will often be used herein. Any reference to developing or otherwise adjusting settings corresponds to a disclosure of adjusting or otherwise developing map data and vice versa unless otherwise noted.

In these fitting and/or mapping sessions, various measurements are taken of the recipient. Typically, these measurements are taken in coordination with stimulation applied to the recipient. Indeed, many of these measurements are measurements of physiological reactions that result from the applied stimulation. These measurements can be evaluated or otherwise used to determine adjustments to the map or otherwise develop new map settings.

In at least some exemplary scenarios, the various measurements are taken while the recipient is awake. In at least some exemplary scenarios, the recipient is dedicated himself or herself to the associated testing or otherwise the associated efforts that results in enabling the measurements to be taken that have utilitarian value with respect to developing settings for the cochlear implant. Indeed, in at least some exemplary scenarios, the recipient is involved in a fitting session or a mapping session or a map development session specifically arranged and set up for that purpose. In an exemplary scenario, at least 30, 40, 50, 60, 70, 80, or 90% if not all of the recipient's cognitive ability at any given time and/or on average (mean, median, and/or mode) is dedicated to the map development process. In an exemplary scenario, the recipient is totally conscious at all times during the session that the recipient is involved in a map development session. Still further, in an exemplary scenario, the recipient is able to stop the map development session at any time upon a conscious decision that no further testing or measurements shall proceed and action upon that decision. Moreover, in at least some exemplary scenarios, the recipient is in a non-tired and/or a non-resting state during the map development session. Indeed, best practices tend to suggest that a recipient come to a map development session or a fitting session well rested, well fed, and before expending typical energy associated with functioning during a day. Also, in an exemplary scenario, objective tests that are relatively time-consuming can be executed during the map development sessions and/or fitting sessions, with the recipient in the aforementioned states detailed above.

Conversely, in at least some exemplary embodiments, map development sessions are at least in part executed while the recipient is sleeping. In an exemplary situation, the clinic where the recipient would normally go for a mapping and/or fitting session has time restrictions and may not be able perform all tests on an individual recipient. At least some exemplary embodiments utilize the many hours that exist during recipient nighttime periods and many recipient nights in between clinic visits in which to collect data or otherwise execute measurements to obtain data that can be utilitarian with respect to developing maps or otherwise fitting or refitting the prosthesis. It is briefly noted that the phrase “recipient night” refers to the equivalent for a given recipient of what is traditionally considered night—a period of rest—as opposed to the celestial phenomenon where the sun is not visible. By way of example only and not by way of limitation, a recipient who works during periods of darkness and rests during periods of light (i.e., a night shift worker) would have a recipient night occurring during sunlight. Any reference to night herein refers to something associated specifically with the recipient as opposed to the celestial phenomenon unless otherwise noted.

In at least some exemplary embodiments, at least some objective measurements or other measurements are executed prior to a clinic visit and/or between clinic visits but before the next visit. This can have utilitarian value with respect to allowing or otherwise enabling the clinician to have more time to obtain more information with less or little or no time spent conducting the testing or otherwise executing the measurement methods that can have utilitarian value with respect to developing map data or otherwise adjusting the prostheses relative to that which would otherwise be the case without the teachings detailed herein. In at least some exemplary embodiments, measurements are executed while the recipient is sleeping and/or in close temporal proximity to sleep periods of the recipient.

In at least some exemplary embodiments associated with a method of utilizing or otherwise adjusting or otherwise mapping a hearing prosthesis, clinics may not be able to assess individual channels in detail. Conversely, in at least some exemplary embodiments, by utilizing the teachings detailed herein, the teachings can enable mapping parameters to be optimized on a per channel basis using results from the objective measures, which measures are implemented in accordance with the teachings detailed herein. “Bad” channels and/or device failures could also be detected early, prior to clinical signs, in at least some exemplary embodiments implementing the teachings detailed herein.

In this regard, at least some exemplary embodiments include executing measurements, including objective measurements, while the recipient is sleeping and/or in close temporal proximity to periods where the recipient is sleeping. Embodiments also include monitoring whether a recipient is asleep, monitoring the stage of sleep in which is the recipient, and/or performing measures, including objective measures, utilizing a prosthesis, such as a hearing prosthesis such as a cochlear implant (CI) during recipient nighttime/while the recipient is asleep/in close temporal proximity to the sleep period of the recipient.

At least some exemplary embodiments include executing method of measurement on a recipient while the recipient is sleeping. In an exemplary embodiment, stimulation can be applied to the recipient so as to evoke physiological reactions to this stimulus, which physiological reactions can be measured. In at least some exemplary embodiments, while the recipient is sleeping, the recipient's auditory pathway, continues to register and process stimulation, such as by way of example, sounds, albeit at least in some exemplary embodiments on a basic level.

At least some exemplary embodiments include performing objective measures at sub-audible levels. Other exemplary embodiments include performing objective measures utilizing stimuli that could be audible and/or otherwise is audible. In some exemplary embodiments, such stimuli are presented in a way to not disturb sleep or otherwise in a manner that reduces the likelihood that sleep will be disturbed relative to that which would otherwise be the case, all other things being equal. Indeed, in an exemplary embodiment, methods include, implementing stimulation that is part of a soothing and/or desired or otherwise pleasing stimulus. This combined with, in some other embodiments, monitoring a sleep state of the recipient (although in some other embodiments, the two are not combined—the two can be executed separately or only one is executed or only the other is executed—as noted below, embodiments include executing one or more method actions detailed herein at the exclusion of one or more other method actions detailed herein) and/or the modulation of stimuli to avoid wakefulness and/or to be incorporated into a wake-up alarm sound (more on this below). The soothing stimulus could be, by way of example only and not by way of limitation, a tinnitus masker stimulus or other type of soothing sound or otherwise an oculus background sound (rain sound, ocean sound, fan sound, jet noise sound, etc.). The stimulus utilized in at least some exemplary embodiments could be an audiobook and/or the wake-up alarm could be the objective measure stimulus. These exemplary scenarios are described in greater detail below.

At least some exemplary embodiments include utilizing the objective measures to understand or attempt to understand the underlying physiological processes following implantation of a hearing prostheses, such as a cochlear implant. Results could also be used to recommend or otherwise identify and/or implement map adjustments either by the clinic or outside of the clinic in an automated fitting application. Some exemplary embodiments utilizing one or more or all of the teachings detailed herein can enable the allowance of additional test intervals than the typical clinic schedule, all other things being equal. In at least some exemplary embodiments, more frequent map adjustments could impart benefits faster and/or allow for easier adaptation to smaller, step-wise changes. Diagnostics collected in the recipient nighttime fitting could supplement testing that the clinic performs to enhance and streamline clinic care, again, all other things being equal.

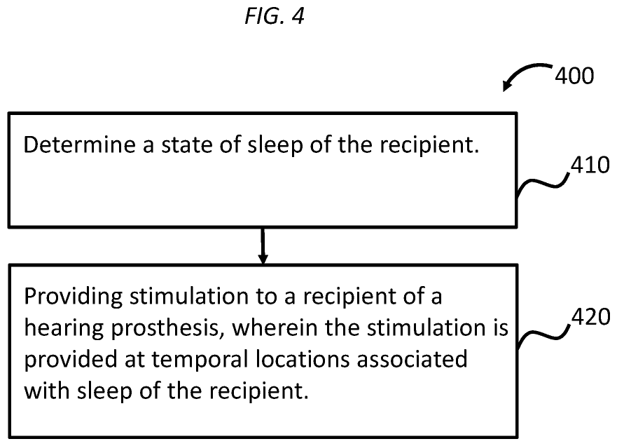

FIG. 4 presents an exemplary flowchart for an exemplary method, method 400, that includes method action 410, which includes determining a sleep state of the recipient. Additional details of this are described below, both with respect to the species of sleep state amongst the genus of sleep state, as well as methods and/or devices and/or systems to execute method action 410. Method 400 further includes method action 420, which includes providing stimulation to the recipient of the hearing prostheses, wherein the stimulation is provided at temporal locations associated with sleep of the recipient. By temporal locations associated with sleep of the recipient, it is meant the temporal period while the recipient is sleeping, the temporal period constituting waking of the recipient, and the temporal period precedent sleeping that occurs after which the recipient has readied himself or herself for sleep and engages in one or more pre-sleep rituals, such as reading a book. This as opposed to the temporal period where the recipient is changing from daywear clothing to nightwear clothing, brushing of one's teeth, or activities that occur after the recipient has finally silenced an alarm for a given day. Thus, it is to be understood that a sleep state can include periods where the recipient is conscious or otherwise not sleeping, as long as those states are associated with sleeping.

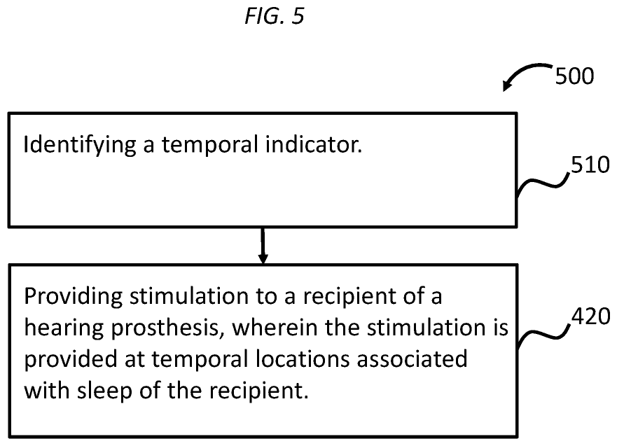

It is noted that method 400 includes method action 410 which includes determining a state of sleep of the recipient. In at least some exemplary embodiments of method 400, there is the action of determining what type of stimulation or otherwise determining that stimulation should be provided based on the results of method action 410. By way of example only and not by way of limitation, if the state of the recipient is in a non-sleep state, stimulation that is for a sleeping recipient will not be implemented. More on this below. That said, it is noted that in an alternate embodiment, there is no determination of a state of sleep of the recipient. Instead, there is a method that entails identifying a temporal indicator, and based on the identification, executing method action 420. Briefly, FIG. 5 depict a flowchart for such a method, method 500, which includes method action 510, which includes identifying a temporal indicator, along with method action 420. In an exemplary embodiment of this embodiment, the temporal indicator is a temporal indicator that is correlated or otherwise statistically significant with respect to the recipient likely sleeping. By way of example only and not by way of limitation, for a person that works a normal 9-to-5 job and otherwise obtains eight hours of sleep between the hours of 10:00 PM and 6:00 AM, the identified temporal indicator could be to 2:00 AM or any other time statistically associated with a given state of sleep (e.g., based on statistical data, all other things being equal, the recipient is typically in stage III or stage IV sleep between the hours of 3:00 AM and 5:00 AM, and thus the identified temporal indicator could be 2:00 AM for stage I or stage II sleep, and 4:00 AM for stage III or stage IV sleep). The point is, method action 410 is not necessary to implement at least some exemplary embodiments.

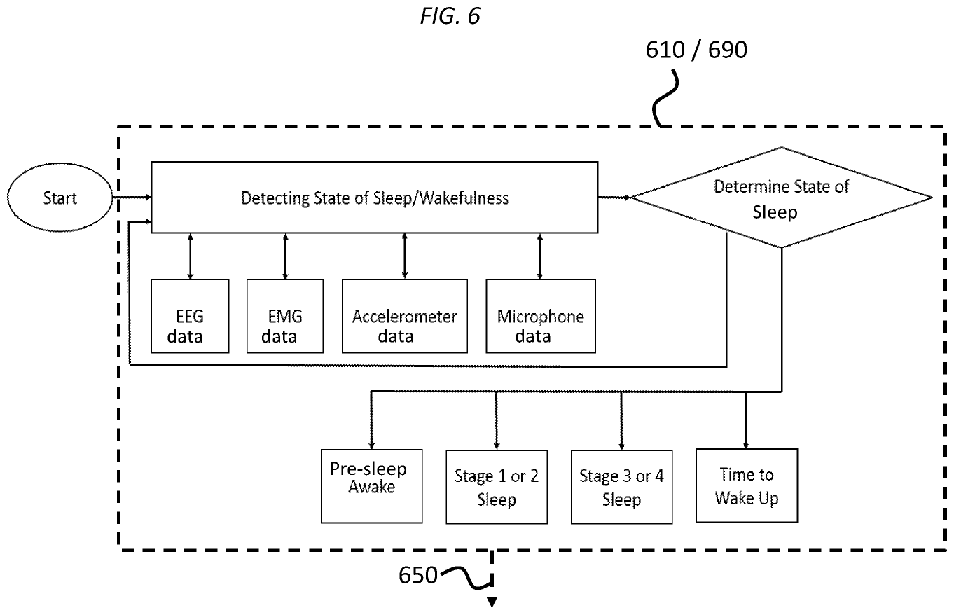

That said, FIG. 6 presents an exemplary flowchart/functional diagram for an exemplary algorithm that is utilitarian with respect to determining a sleep state of the recipient. Also superimposed on that figure is a black-box 610/690, representing a prosthesis 610 corresponding to any of the prostheses disclosed herein or any other that can be the subject of the teachings herein, and representing a separate device or system 690 that is separate from the prosthesis that outputs a signal 650 to a remote system or to the prosthesis indicating the state of sleep. More specifics about these two representations are presented below.

More specifically, an electroencephalogram (EEG) is utilitarian with respect to determining stages of sleep, and in some embodiments, EEG system is utilized to determine a sleep state. In some exemplary embodiments, EEG system is an integral part of the hearing prostheses. Indeed, in an exemplary embodiment, the EEG system utilizes electrodes that are part of the cochlear implant. By way of example only and not by way of limitation, in some exemplary embodiments, the EEG system utilizes the electrodes that are located in the cochlea and/or the return electrodes that are located outside the cochlea, such as the so-called hardball which is typically supported by a separate lead separate from the lead assembly that is for the intracochlear electrodes, but need not necessarily be so, and/or the so-called plate on the housing or otherwise supported by the housing of the receiver stimulator. In an exemplary embodiment, the EEG system utilizes only one or more or all of the aforementioned electrodes in any combination to implement EEG monitoring of the recipient to determine sleep status. That said, in some alternate embodiments, extra electrodes beyond those just detailed are included with the cochlear implant to execute EEG monitoring and otherwise obtain EEG data. Again, in other embodiments, the EEG data is completely separate.

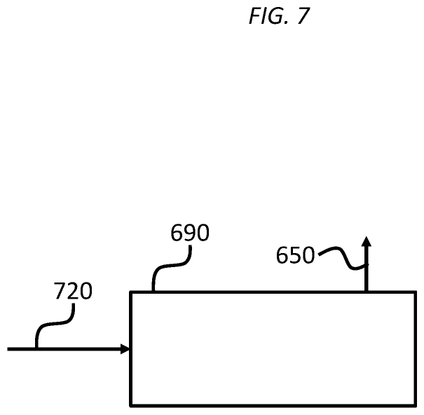

Other embodiments can include along with or without EEG monitoring, an electromyogram (EMG) system to monitor muscle tension, and/or an accelerometer to monitor movement, and/or a microphone to record frequency and/or volume of snoring activity and/or patterns of breathing and/or other sounds. In an exemplary embodiment, the hearing prosthesis 610 and/or the separate device 690 is configured such as with programming or the like, to analyze the data that comes from the EMG system and/or the accelerometer and/or the microphone and/or the EEG system, and based on the analysis, determine a state of sleep of the recipient. In an exemplary embodiment, it is the processor of the cochlear implant or other prostheses, whether implanted or external, that is utilized to execute the analysis. Again, in some other embodiments, a separate device 690 does this. In an exemplary embodiment, the separate device can be a personal computer or a dedicated device that includes a microprocessor that is programmed accordingly. By way of example only and not by way of limitation, this separate device can include a microphone to record the sounds, and/or can be in signal communication with electrodes that are attached to the recipient which may or may not be part of a hearing prostheses, and/or can be in signal communication with an accelerometer that may or may not be part of the hearing prostheses, and/or can be in signal communication with a separate microphone. The device 690 can receive the signals from the various components and analyze the signals to determine the state of sleep. By way of example only and not by way of limitation, FIG. 7 presents an exemplary schematic of input 720, representing input corresponding to EEG data, EMG data, accelerometer data, and/or microphone data, being received by device 690, which again can be a personal computer or a mainframe computer or a smart device, such as for example, a smart phone or a smart handheld device, or any other device that can enable the teachings detailed herein, which may or may not be co-located with the recipient (the device 690 could be located remotely and in signal communication with the devices that generate the input 720 via, for example, wireless technology and/or the Internet, etc.—some additional details of this are below). The device 690 includes processors or includes logic circuitry or the like that is configured to analyze the input and determine a state of sleep and then provide output 650, to, for example, the hearing prostheses directly, or to another device or component that then controls or otherwise activates the hearing prostheses to execute the measurements or other actions detailed herein which are state of sleep dependent actions.

Still, in at least some exemplary embodiments, it is the hearing prostheses as an integrated unit that can determine the state of sleep. As detailed above, in an exemplary embodiment, a cochlear implant can have all of the componentry needed to implement state of sleep determination, or at least the componentry to collect the data needed for state of sleep determination. Thus, in one embodiment, the cochlear implant can be configured to detect that a recipient is asleep and then determine the stage of sleep of the recipient. For instance, the electrodes that are utilized to evoke a hearing percept during normal operation of the cochlear implant, are utilized to monitor the EEG and/or the EMG of the recipient. Still further, in at least some exemplary embodiments, an accelerometer of the hearing prosthesis, which could be implanted in the recipient or could be worn by the recipient outside of the recipient, could be configured to detect movement. Moreover, a microphone of the prosthesis can be utilized to detect sounds of breathing and/or snoring and/or other sounds. With respect to the microphone, in at least some exemplary embodiments, the microphone of the hearing prosthesis can be utilized. Indeed, in at least some exemplary embodiments, the microphone is an implantable/implanted microphone. In this regard, in at least some exemplary embodiments, the detection actions can be executed via a totally implantable hearing prosthesis, with the microphone is implanted beneath the skin of the recipient. In any event, these monitors could be used to continuously and/or periodically assess sleep stages and detect wakefulness. Once the sleep stage is confirmed based on input from the monitors, the cochlear implant can be controlled to perform the objective measures and/or to provide a soothing sound to maintain sleep, in some embodiments.

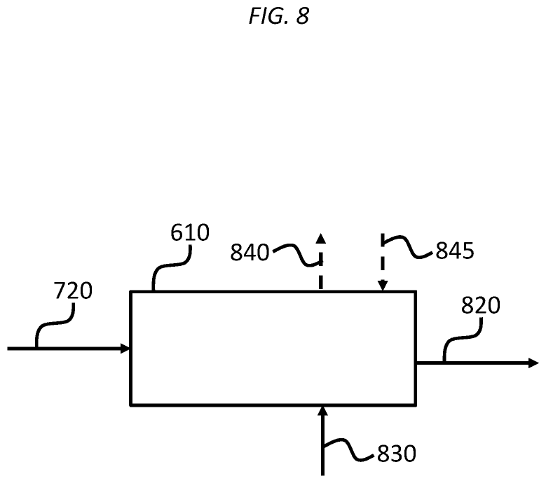

FIG. 8 presents a functional schematic of a prosthesis 610, which can correspond to any of the prostheses detailed herein. As seen, prosthesis 610 receives input 720, corresponding to any of the inputs that can be utilitarian with respect to determining a sleep state of the recipient, such as, for example, signals of the body that represent electrical signals that are detectable by the electrodes of a cochlear implant for EEG and/or EMG purposes, acoustic signals are vibrations that reach the microphone, and/or movement that is detected by the accelerometer. In some exemplary embodiments, the prosthesis 610 is configured to analyze the inputs, and determine a sleep state of the recipient, and then output stimulation 820 in accordance with that determination.

It is seen that 2 dashed arrows extend out of and into prosthesis 610: arrow 840 and arrow 845. These represent, respectively, an alternate embodiment where it is not the prosthesis 610 that determines the state of sleep, what a remote device or a separate device, such as device 690. In this regard, in an exemplary embodiment, the prosthesis 610 is configured to transmit a signal, represented by arrow 840, indicative of the received input 720 to the device 690, where the device 690 analyzes that signal, and then receive a control signal or instruction signal, represented by arrow 845, which instructs the prostheses to generate stimulation 820, which stimulation is utilized to perform testing and measurements. Also, can be seen with respect to FIG. 8 is arrow 830, which represents the measurements that are taken by the prosthesis with respect to, for example, the objective testing, which is based on the stimulation signal 820. Additional details of the objective testing are detail below.

It is also noted that while the embodiments detailed herein are disclosed in terms of utilizing the hearing prosthesis to execute the objective testing/objective measurements, in some alternate embodiments, a separate device is utilized to execute those tests/measurements as well, at least in part. Indeed, in an exemplary embodiment, such as where there is EcoG testing, a separate sound maker/sound generator is utilized that is not part of the hearing prosthesis. Moreover, in some embodiments, the sensors that are utilized for the objective testing are not part of the prostheses. Any device, system, and/or method that can enable the teachings detailed herein can be utilized in at least some exemplary embodiments.

In view of the above, with reference to method action 420, it can be seen that in an exemplary embodiment, the stimulation is part of a measurement method executed at the temporal locations associated with sleep of the recipient. Further, in an exemplary embodiment, the action of providing the stimulation executes measurements of the recipient, such as, for example, objective measurements, and further, the temporal locations are temporal locations corresponding to at least one of the recipient in a going to sleep state, the recipient asleep or the recipient just waking up. By way of example, in an exemplary embodiment, the cochlear implant can perform objective measures during periods of Stage 3 and/or 4 sleep. Once the system has determined that stage 3 or 4 sleep has been reached (or once a determination that statistically speaking, such has been reached), supra-threshold stimulation levels can be used. In an exemplary embodiment, the stimulation levels are under a given threshold of loudness. In an exemplary embodiment, the implant or other device monitors the EEG, EMG, accelerometer, and/or microphone for signs of wakefulness. In an exemplary method, testing stops if the system determines that there are one or more signs in the data indicative of wakefulness or that the recipient is beginning to transition from one of the stages of sleep. In an exemplary embodiment, if there are consistent signs of wakefulness, the prostheses would reduce the volume of the stimulus and find the level where sleep is maintained, or otherwise stop testing. With regard to this point, in an exemplary embodiment, the cochlear implant or the device or any other device can be configured to execute the teachings herein in an iterative or in an intelligent manner so that the stimulation is adjusted to maintain the sleep state of the recipient or otherwise to avoid waking the recipient. Indeed, in an exemplary embodiment, it is to be understood that the teachings detailed herein utilize a continuous or periodic feedback loop where the stimulation is adjusted, including completely stopped, based on the data obtained from the monitors, which data is indicative of the state of sleep of the recipient.

Accordingly, again referring to method action 420, in at least some exemplary embodiments, the stimulation is audible, and one of the temporal location corresponds to the recipient being asleep. Still further, as can be seen, in an exemplary embodiment, there is the action of automatically determining a sleep state of the recipient using the hearing prosthesis (although in other embodiments, as noted above, the hearing prosthesis is not used, and in other embodiments, another device is used in conjunction with the hearing prosthesis), and automatically controlling the hearing prosthesis to provide the stimulation based on the determination.

It is noted that testing may also be performed just prior to sleep and/or when waking up, the latter potentially in at least some exemplary embodiments enabling for louder stimulation and objective measures that require attention. By way of example only and not by way of limitation, there is a statistically significant group of people that will often read prior to falling asleep in the pre-sleep period. In an exemplary embodiment, the hearing prosthesis could present an audio book with embedded stimuli that are predictable and measurable by AEPs (e.g. P1, MMN, CAEPs—more on this below). Alternatively, and/or in addition to this, in an exemplary embodiment, the prosthesis could use soothing stimuli such as white noise or a tinnitus suppression stimulus to help people fall and stay asleep while also delivering predictable components for objective measures. Conversely, wake up alarms built into the device could be used to conduct objective measures at increasing levels (e.g. NRT). In such a situation in some exemplary embodiments, the level would be such as to be a level that statistically speaking, should and actually does wake the recipient at some point (in some embodiments, the level is comfortable, in others, it is not). The recipient could set the time and the loudest volume for the wake-up alarm before going to sleep. The system can thus combine the loud stimulation—the stimulation having magnitudes which, statistically speaking, would wake the recipient, or otherwise which would be less than utilitarian when applied during sleep because, statistically speaking, it might wake the recipient—with activities corresponding to that which are intended to wake up the recipient, such as an alarm.

Thus, it can be seen that in an exemplary embodiment, there is the method action of automatically determining various sleep states of the recipient (pre-sleep, stage 3 or 4, etc.), and variously applying the stimulation at sub-threshold levels and supra-threshold levels based on the automatic determinations of the various sleep states. Further, it is noted that in some exemplary embodiments, the stimulation is part of a measurement method executed at the temporal locations associated with sleep of the recipient, and the stimulation is provided at an audible level and measurements are taken based on the stimulation while the recipient is in a going to sleep state and a sleep state. As with any embodiment detailed herein, in an exemplary embodiment, the hearing prosthesis is a cochlear implant.

Still further, in an exemplary embodiment where the stimulation as part of a measurement method executed at temporal locations associated with sleep of the recipient, the action of performing measurements is executed without association with an audiologist or a hearing clinician, and, in some embodiments but not others, without any other healthcare professional.

Exemplary embodiments include utilizing the measurements/the results of testing to fit or refit the hearing prosthesis. In an exemplary embodiment, as will be described in greater detail below, the map is adjusted, and/or a new map is developed based on the measurements obtained during the temporal locations associated with sleep of the recipient. In an exemplary embodiment, there is thus the action of fitting or refitting the hearing prosthesis based at least in part on the measurement method. In an exemplary embodiment, the action includes automatically fitting or refitting the hearing prosthesis, concomitant with the previous paragraphs disclosure of taking actions without association with an audiologist and/or a hearing clinician and/or any healthcare professional (although with respect to the latter, in at least some exemplary embodiments, a sleep healthcare professional may be utilized to monitor the sleep states or otherwise to get the recipient in a state of sleep—this could be utilitarian with respect to infants or old people—the point is that in some embodiments, the teachings detailed herein vis-à-vis the monitoring and measurements can be executed without a hearing professional but because these measurements are associated with sleep, it is possible that a healthcare professional can be involved). That said, as will be detailed below, in some alternate embodiments, the data that is collected via the execution of the methods are provided to a healthcare professional, such as a hearing professional, which are analyzed and utilized by such to fit or refit the hearing prosthesis.

The measurements executed according to the teachings herein, such as the objective measures, can be used outside of the clinic in an automated fitting arrangement. Some examples of embodiments entail the action of fine tuning of the map after the initial map set up by the audiologist, depending on the results of the measures taken associated with sleep of the recipient, T and/or C levels could be modified, resulting in improvement of, for example, objective measure result (such as MMN—more on this below). Indeed, in an exemplary embodiment, this also results in improvement of speech understanding resulting from hearing percepts utilizing the cochlear implant. In an exemplary embodiment, a speech understanding score on a speech understanding test (a standardized test, or a uniform apples to apples test that can gauge performance/improvement) can be increased by at least 10, 15, 20, 25, 30, 35, 40, 45, or 50 percent or more. Moreover, relatively small changes made to the map, which are made over time, can, in some instances, ease adaptation for the recipient.

Moreover, the teachings detailed herein can be executed such that the measurements/testing could also gather data starting immediately after activation. In some embodiments, the measurements/testing gathers data on the day of activation, 1, 2, 3, 4, or 5 days after, etc.). Such can have utilitarian value with respect to obtaining information about the acclimation and/or changes over the first few months. In some embodiments, these measures could provide information about the physiological development following implantation of the prosthesis. Indeed, in some embodiments, recipient nightly measurements are used to track and/or quantify neural changes acutely after activation and then over time and/or classify recipients based on their rate and/or degree acclimation. Also, stimulation at night is used in some embodiments to provide conditioning to improve impedances upon waking up.

Accordingly, in an exemplary embodiment, there is a method that includes activating a cochlear implant or other hearing prosthesis for the first time after implantation (depending on the implantation philosophy, some wait two weeks after implantation, while others wait one month after implantation, etc.). Further, the actions of measuring or otherwise taking objective measurements are executed, outside of the clinic and/or after the recipient leaves the clinic after activation for the first time of the prosthesis, within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, or 75 days of the time of first activation and/or within the time that the initial map has been loaded into the prostheses for use by the recipient in normal life (i.e., the finalized initial fitting). In an exemplary embodiment, any one or more the method actions detailed herein, such as the actions of taking any one or more of the objective measurements detailed herein, are executed in at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, or 75 days of the aforementioned time (e.g., every day, every other day, every day except Sunday or Saturday or Friday (holy days depending on a given religion, for example), days of rest excluded, days of hard partying excluded (e.g., Friday and Saturday nights have irregular, if any, sleep)). In an exemplary embodiment, adjustments can be made to the prosthesis on a basis corresponding to any of the aforementioned temporal examples. In some embodiments, this is done without intervention by a healthcare professional and/or audiologist or hearing professional and/or without having to participate in testing directed by or otherwise under the control of a health care professional and/or an audiologist and/or a hearing professional. To be clear, embodiments also extend to temporal periods beyond that detailed above. Moreover, the aforementioned temporal periods can be keyed to other dates, including arbitrary dates, such as a date after 1, 1.5, 2, 2.5, 3, 3.5, 4, 4.5, 5, 6, 7, 8, 9, 10 or 15 or 20 or 30 or 40 or more years after first activation of the device (or the initial device, in the event that the device was replaced), etc. Indeed, in some embodiments, the teachings herein can be used over a lifetime with a hearing prosthesis, and thus can be executed over a period of days, weeks or months or 0.5, 0.75, 1, 1.5, 2, 2.5, 3, 3.5, 4, 4.5, 5, 6, 7, 8, 9, 10 or 15 or 20 or 30 or 40 or more years after first activation of the device (or the initial device, in the event that the device was replaced), etc.

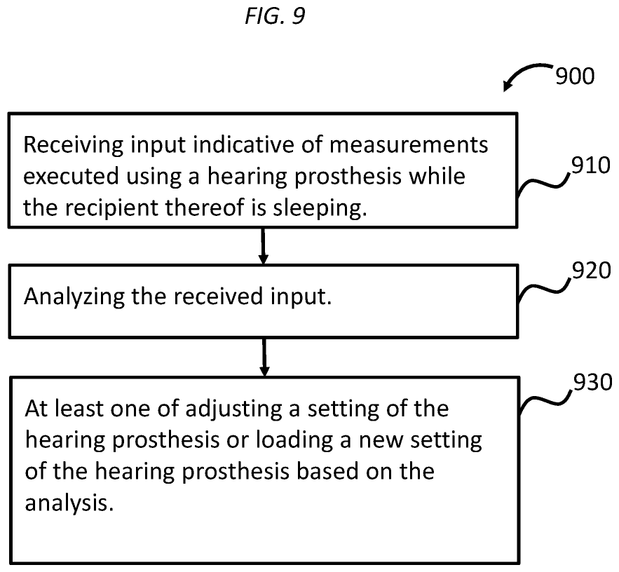

In view of the above, FIG. 9 presents an exemplary algorithm for an exemplary method, method 900, which includes method action 910, which includes receiving input indicative of measurements executed utilizing a hearing prosthesis while the recipient thereof is sleeping (sleeping, as opposed to pre-sleep or the wake-up period). This method action can be executed by executing any of the teachings detailed herein that can enable such or other variations thereof, or by utilizing any other device, system and/or method that will enable method action 910 to be executed. Method 900 further includes method action 920, which includes analyzing the received input. In an exemplary embodiment, this can be performed utilizing a computer program in an automated manner. By way of example only and not by way of limitation, this can be executed utilizing a personal computer and/or a smart phone or smart device and/or even the hearing prosthesis in some exemplary embodiments, where these components are programmed to analyze the received input. In an exemplary embodiment, the input is received from a remote location. In this regard, by way of example only and not by way of limitation, the hearing prosthesis or the devices under the control of the recipient or otherwise in the possession of the recipient obtain the data indicative of measurements executed while the recipient is sleeping, and provide a data package or the like to a remote location, such as an audiologist center or a healthcare professional center or the like, where action is executed. In an exemplary embodiment, the input is received by a device that is under the control or within the possession of the recipient, and that device executes method action 920.

Method 900 also includes method action 930, which includes at least one of adjusting a setting of the hearing prosthesis or loading a new setting of the hearing prosthesis based on the analysis. In an exemplary embodiment, a feature of the map that is currently in the hearing prosthesis is adjusted, while in another exemplary embodiment, the map is completely replaced with a new map (or a new map is added—the old map can be retained). Any arrangement that can enable the execution of method action 930 can be utilized in at least some exemplary embodiments. Further, in an exemplary embodiment, after method action 930 is executed, there is the action of utilizing the hearing prosthesis to evoke a hearing percept utilizing the adjusted setting and/or the newly loaded setting.

Concomitant with the teachings detailed above, in an exemplary embodiment, method actions 920 and/or 930 are executed automatically. For example, method action 930 can be executed automatically based on the analysis of method action 920. Still, in alternate embodiments, these are done under the direction and control via affirmative actions by healthcare professional or the like. Moreover, in some embodiments, it is possible that the recipient himself or herself can execute method action 930.

Again, as noted above, teachings herein are applicable to a prosthesis that is in the form of a cochlear implant. In this regard, by way of example, method action 930, once executed, or when executed, is executed such that it causes the cochlear implant to stimulate the recipient consistently in a different manner for a given input than that which would have been the case in the absence of the adjusting or loading, all other things being equal. In an exemplary embodiment, for a given sound input, e.g., a sine wave at 700 Hz with at 60 dB fed directly into the sound processor of the prosthesis bypassing the microphone, the output of the hearing prosthesis will be different than that which would have been the case prior to method action 930. This as opposed to merely changing the volume or the like of the prosthesis.

Again, as noted above, the measurements of method action 910, more accurately, the measurements upon which method action 910 is based, are objective measurements of the recipient (again, more on this below).

In an exemplary embodiment, method action 930 is executed 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, or 75 times or any range of values therebetween (e.g., 17 to 55) within the aforementioned time from initial activation (e.g., every day, every other day, every day except Sunday or Saturday or Friday, etc.). In an exemplary embodiment, this is done without intervention by a healthcare professional and/or audiologist or hearing professional and/or without having to participate in testing directed by or otherwise under the control of a health care professional and/or an audiologist and/or a hearing professional.

Consistent with the theme that the teachings detailed herein can be utilized in an automated fashion and/or without a clinician and/or in close temporal proximity to initial activation of the prosthesis, in an exemplary embodiment, the actions of adjusting or loading of method action 930 are executed automatically in real time with the execution of the measurements. In an exemplary embodiment, the actions of adjusting or loading of method action 930 are executed within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 or 60 or 72 or 96 or 100 hours or any value or range of values therebetween (e.g., 17 to 80, 99, 55) from the time that the measurements are taken and/or from the time that the input is analyzed.

Again, in some embodiments, the analysis and the adjusting and loading are executed independently of a healthcare professional. In other embodiments, this is not the case. In an exemplary embodiment, these could instead or in combination (e.g., some automatically, some by the audiologist) be executed by the audiologist or other healthcare professional. Further, for example, the measurements can be completed without the audiologist, such as independently by the recipient and/or caregiver, and then sent to the audiologist or other healthcare professional for evaluation. In some embodiments, no changes are automatically made. The audiologist makes changes based on the results of the measurements, after evaluation thereof In other embodiments, the audiologist is completely out of the loop with respect to a given adjustment.

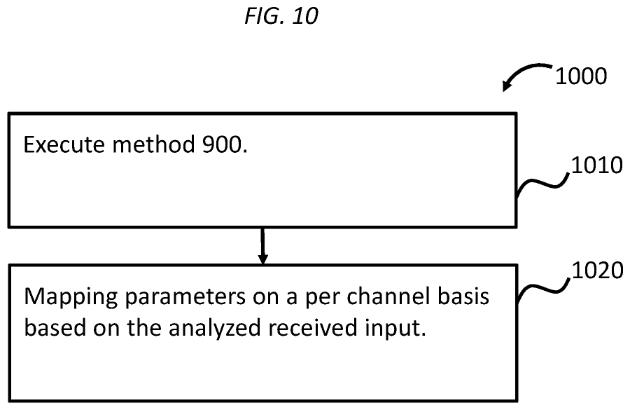

As noted above, some embodiments of the hearing prosthesis to which the teachings herein are applicable are prostheses that include a plurality of channels. By way of example only and not by way of limitation, in an exemplary embodiment, a cochlear implant is included with filters that divide up a sound frequency spectrum into channels (e.g., channels 1-22), of separate frequency ranges. One channel, or more accurately, the sound falling within one frequency band associated with one channel can be processed in a different manner than the sound falling within another frequency band associated with another channel, in some instances. Accordingly, in an exemplary embodiment, there is an exemplary method, method 1000, as represented by the flowchart on FIG. 10, which method includes method action 1010, which includes executing in whole or in part method 900. Method 1000 further includes method action 1020, which includes mapping parameters on a per channel basis based on the analyzed received input received at method action 920 of method 900. This as opposed to mapping parameters on a multichannel basis. In an exemplary embodiment, the action of adjusting or loading results in a change to a channel of the hearing prosthesis and no change to another channel of the hearing prosthesis. Note further that in an exemplary embodiment, the action of adjusting or loading results in the elimination of one or more channels as utilized by the hearing prosthesis to evoke a hearing percept.

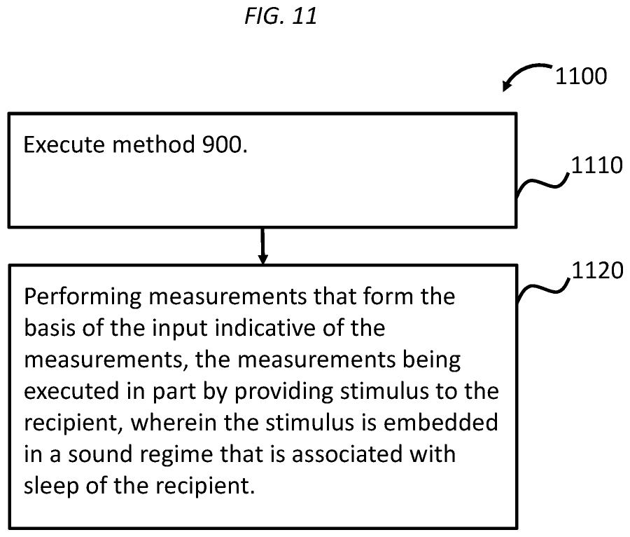

In an exemplary embodiment there is another method, method 1100, as represented by the flowchart on FIG. 11, which includes method action 1110, which includes executing in whole or in part method 900. Method 1100 further includes method action 1120, which includes the action of performing measurements that form the basis of the input indicative of the measurements, the measurements being executed in part by providing stimulus to the recipient, wherein the stimulus is embedded in a sound regime that is associated with sleep of the recipient.

It is briefly noted that method 1100 makes clear that the method actions detailed herein, as disclosed, are not necessarily disclosed in the order in which they are executed. In this regard, in at least some exemplary embodiments, method action 1120 would be executed before method action 1110. Accordingly, unless otherwise noted, any sequence of presentation of method actions herein does not correspond to a requirement that those method actions be presented in that sequence. Any sequence that can enable the teachings detailed herein can be utilized in at least some exemplary embodiments. That said, any disclosure herein of method actions presented in a sequence corresponds to a disclosure of those method actions being practiced exactly in that sequence.

In an exemplary embodiment, the stimulus is embedded in the sound regime that is presented while the recipient is sleeping, such as while the recipient is in stage I and/or in stage II and/or in stage III and/or in stage IV sleep.

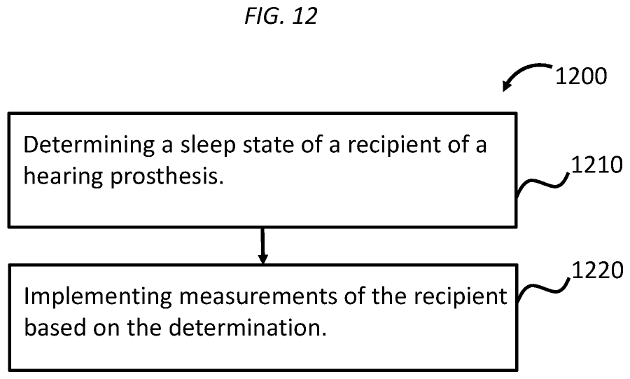

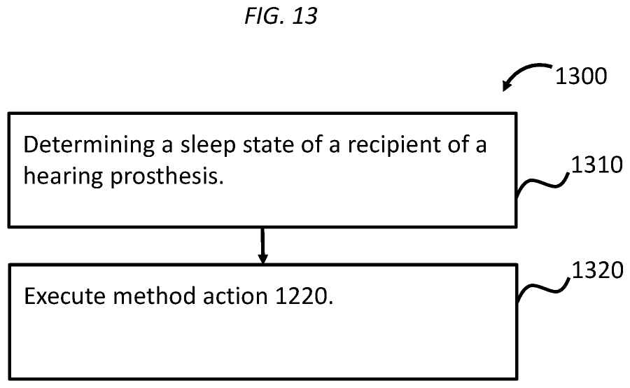

FIG. 12 presents an exemplary algorithm for an exemplary method, method 1200, that includes method action 1210, which includes the action of determining a sleep state of a recipient of a hearing prosthesis. In an exemplary embodiment, this is executed in accordance any of the teachings detailed herein or any other manner that will enable this method action. Method 1200 also includes method action 1220, which includes implementing measurements of the recipient based on the determination, which is again a method that can be executed in accordance to any of the teachings detailed herein or any other teachings they can have utilitarian value. It is briefly noted that in a variation of method 1200, method action 1210 instead entails determining a feature indicative of a sleep state of a recipient of a hearing prostheses. By way of example only and not by way of limitation, this can entail determining a time in conjunction with statistically significant data indicating that the recipient is likely sleeping. Thus, in an exemplary embodiment, there is a method 1300, which is represented by way of example in the algorithm of FIG. 13, which includes method action 1310, which includes determining a feature indicative of a sleep state of a recipient of a hearing prosthesis. Method action 1310 can be executed by executing method action 1210 or by the temporal method noted, or by any other activities that can enable this action. Method 1300 also includes method action 1320, which includes executing method action 1220.

At this time, it is noted that some embodiments include programming that can enable the execution of one or more of any method action detailed herein. Accordingly, it is briefly noted that in an exemplary embodiment, there is a non-transitory computer readable medium having recorded thereon, a computer program for executing a method, the program including code for determining a feature indicative of a sleep state of a recipient of a hearing prosthesis and code for implementing measurements of the recipient based on the determination. That is, there is code for executing method 1300, just as there is code for executing method 1200 or any other method or method action detailed herein. Corollary to this is that in at least an exemplary embodiment, the aforementioned medium includes code for analyzing input indicative of the sleep state of the recipient, wherein the code for determining a feature indicative of a sleep state is code for determining the sleep state of the recipient that uses the analysis of the input indicative of the sleep state of the recipient. This is, in essence, code for executing method 1200 and additional method actions, such as the method action of analyzing input indicative of the sleep state of the recipient. Hereinafter, the teachings below will be described for the most part in terms of method actions, but it is to be noted again that any disclosure of a method action corresponds to a disclosure of a medium having code for executing that method action providing that the art enable such.

In an exemplary embodiment, there is expanded method 1200 or method 1300, which includes the action of analyzing a first input indicative that the recipient is in a first sleep state. This can be the pre-sleep state, the sleep state, or any of the species thereof (Stage I-IV), or the wake-up state. In an exemplary embodiment, the action of determining a feature indicative of a sleep state includes determining the sleep state of the recipient by determining that the recipient is in the first sleep state based on the analysis of the first input. Further, in an exemplary embodiment, the method includes the action of, based on the determination that the recipient is in the first sleep state, automatically implementing a first objective measurement regime from amongst a plurality of measurement regimes, thus executing the action of implementing the measures of the recipient. (Again, some additional examples of the objective measurements will be described below in greater detail.)

Expanding upon the just detailed expanded method, in an exemplary embodiment, there is the additional method action of analyzing a second input indicative that the recipient is in a second sleep state, wherein determining the sleep state of the recipient includes determining that the recipient is in the second sleep state based on the analysis of the second input. Also, there is the additional method action of, based on the determination that the recipient is in the second sleep state, automatically implementing a second objective measurement regime from amongst the plurality of measurement regimes, thus executing the action of implementing the measures of the recipient, wherein the second objective measurement regime is different from the first objective measurement regime, and the second sleep state is different from the first sleep state.

In a variation of the above method, in an exemplary embodiment, there is the additional method action of analyzing a second input indicative that the recipient is still in the first sleep state, wherein determining the sleep state of the recipient includes determining that the recipient is in the first sleep state based on the analysis of the second input. Also, there is the additional method action of, based on the determination that the recipient is in the second sleep state, automatically implementing a second objective measurement regime from amongst the plurality of measurement regimes, and/or continuing to implement the first objective measurement regime thus executing the action of implementing the measures of the recipient, wherein the second objective measurement regime is different from the first objective measurement regime.

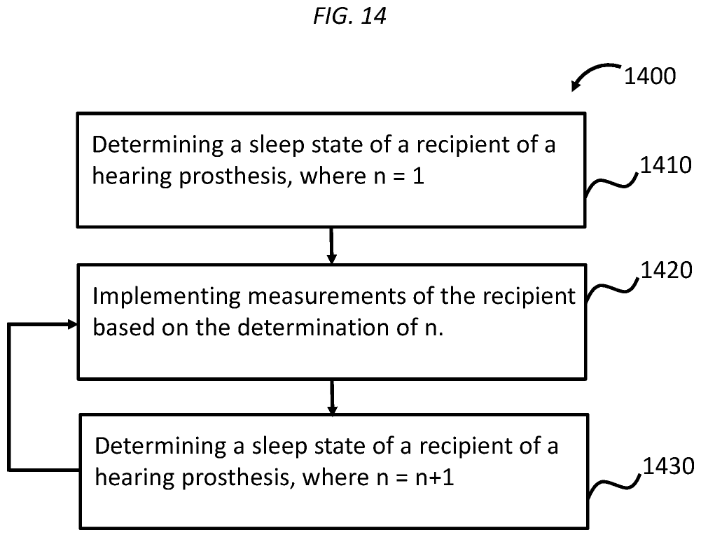

Briefly, FIG. 14 presents an exemplary algorithm for an exemplary method, method 1400, which shows the repetitious nature of some of the teachings detailed herein. In this regard, method 1400 includes method action 1410, which includes determining a sleep state of the recipient of a hearing prosthesis, where n=1. This could be the first determination. This could be the tenth determination for that matter, as n is simply utilized as a counter for at least a portion of the method, as opposed to the counter for the entire method. Method 1400 proceeds to method action 1420, which includes implementing measurements of the recipient based on the determination of n. If the determination is that the recipient is in the first or second stage of sleep, the measurements deemed appropriate for that stage of sleep are applied. If the determination is that the recipient is in the third or fourth stage of sleep, the measurements deemed appropriate for that stage of sleep are applied, etc. Method 1400 further includes method action 1430, which includes again determining a sleep state of the recipient of a hearing prosthesis, except this time, n=n+1. This can correspond to the second time (or the 11^{th} time) that this occurs. The method then returns to method action 1420, which includes implementing measurements of the recipient based on the determination of now n=2. This goes on and on and on until a determination of a state of sleep is made that no longer applies to the implementation of measurements (e.g., the recipient is fully awake—has permanently shut off the alarm clock/no more snooze button). In an exemplary embodiment, method 1400 is executed for an ultimate value of n of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 125, 150, 175, 200, 250, 300, 350, 400, 450, 500 or more or any value or range of values therebetween in 1 increments, all per 24 hour period/per recipient night/per continuous recipient temporal period associated with sleeping. In an exemplary embodiment, different measurements are implemented for the same sleep state. By way of example only and not by way of limitation, if the sleep state determined for n=33 to 66 is Stage III sleep, the same measurements can be applied for each of the actions 1420 for all of the n values, or different measurements can be applied for some or all of the n values.

In any event, continuing further with the method under explanation above, in an exemplary embodiment, there is the action of analyzing a third input indicative that the recipient is in a third sleep state, wherein the action of determining the sleep state of the recipient includes determining that the recipient is in the third sleep state based on the analysis of the third input (but again, in other embodiments, the third input could lead to a determination of the recipient is still in the first sleep state or the second sleep state, etc.) Further, based on the determination that the recipient is in the third sleep state, the method includes the action of automatically implementing a third objective measurement regime from amongst the plurality of measurement regimes, thus executing the action of implementing the measures of the recipient, wherein the third objective measurement regime is different from the first objective measurement regime and the second objective measurement regime, and the third sleep state is different from the second sleep state and the first sleep state (although in other embodiments, this is not the case as noted). In an exemplary embodiment, the third sleep state can be the first sleep state where the second sleep state is different from the first of the third sleep state.

It is noted that an exemplary embodiment includes methods along the lines of the above method for a fourth, fifth, sixth, seventh, eighth, ninth, 10^{th}, etc., iteration, or for an nth iteration. Where the sleep state can be the same as a prior sleep state or different for a given iteration, and/or where the stimulation and/or testing can be the same as a prior stimulation and/or testing or different for a given iteration.

By way of exemplary scenario only and not by way of limitation, in an exemplary embodiment, a first sleep state can be the pre-sleep state, where the measurements are based on stimulation that is embedded in an audiobook white noise etc., while the recipient is sleeping. Further, a second sleep state can be stage I and/or stage II sleep, where the stimulation is provided at a relatively low level and/or in an inaudible level, in keeping with the fact that the recipient could be woken relatively easily based on relatively low magnitude noises. Further, a third sleep state can be stage III and/or stage IV sleep, with a stimulation is provided at a relatively higher level, in keeping with the fact that the recipient can tolerate relatively higher magnitude noises without being woken. Note also that in an exemplary embodiment, a fourth sleep state to be a determination that the recipient has transitioned from the stage III and/or stage IV sleep to stage I and/or stage II sleep, and thus the stimulation provided would be back to the lower level. Again, teachings detailed herein include the actions of monitoring the sleep state of the recipient and actively managing the stimulation applied to the recipient in response to the monitoring, which can include reducing the magnitude of stimulation upon a determination that the recipient might be “wakened” by the noise or even has been wakened by the noise. With regard to the latter scenario, in an exemplary embodiment, upon a determination that the recipient has woken from a deep sleep, the stimulation is halted until a determination is made that the recipient has fallen back to sleep (people periodically wake in the middle of the night—teachings detailed herein, in some embodiments, address this phenomenon and thus manage the stimulation applied to increase the likelihood that the recipient will fall back to sleep relative to that which would otherwise be the case, all other things being equal). Again, this is concomitant with the features associated with embodiments where there is active management of the stimulation based on active/real-time input indicative of the sleep state of the recipient.

Some exemplary embodiments take in to account the type of sounds that disturb sleep is correlated to factors such as the stage of sleep in which is the recipient, the time of recipient night, and/or specific recipient feelings about the sounds themselves. In at least some exemplary embodiments, noises are more likely to wake a recipient from a light sleep (stage I and stage II) than from a deep sleep (stages 3 and 4) and tend to be more disruptive in the second half of the recipient night. Accordingly, some exemplary embodiments are implemented in a manner where the stimulus is refrained or otherwise never presented during stage I and/or stage II and/or the magnitude and/or frequency (repetition, not sound frequency) and/or duration of the stimulation is more limited than that which would be the case during stage III and/or stage IV. Embodiments include avoiding utilizing sounds that are relevant or more relevant to the recipient and/or or emotionally charged relative to that recipient. In an exemplary embodiment, the stimulus is combined with white noise which can help to maintain sleep by reducing the difference between background sounds and a “peak” sound, like a door slamming. In an exemplary embodiment, this can provide an increased likelihood that the recipient will sleep through the stimulus in an undisturbed or less disturb manner relative to that which might otherwise be the case, all other things being equal. Note that much of this is relative. Indeed, in an exemplary embodiment, infants can be subjected to noises that might be unacceptable to adults while sleeping, such as the sound of a vacuum. That is, the noise that is applied during at least some of the stimulations could be analogous to or the same as the sound of the vacuum, both in frequency and in magnitude, depending on the specific recipient. In this regard, it is noted that in at least some exemplary embodiments, the method actions detailed herein are applicable to infants. In an exemplary embodiment, the method actions detailed herein are applied to human beings who are less than 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 months old or any value or range of values therebetween.

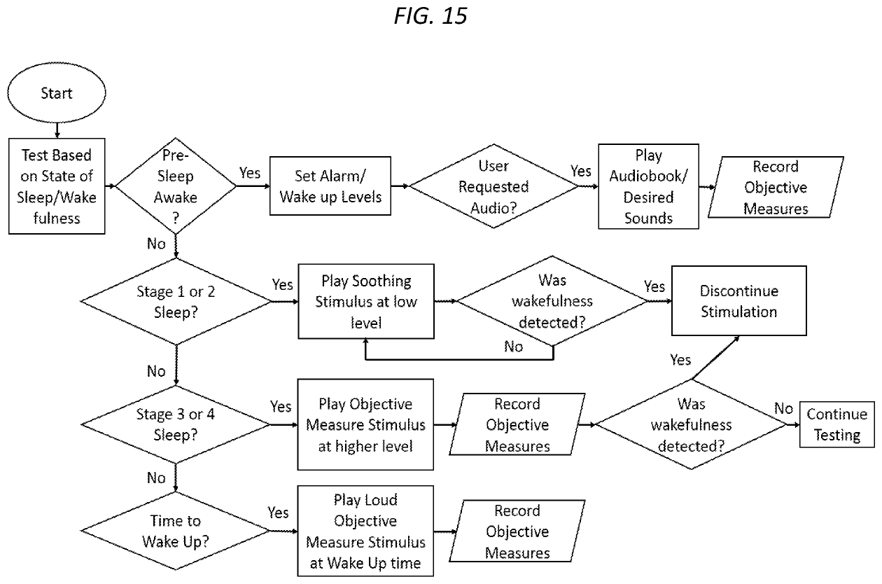

FIG. 15 presents an exemplary algorithm for an exemplary method that can be utilized to determine whether or not testing should be commenced and/or whether or not testing should be continued. This algorithm also presents a flow diagram with respect to the recordation of objective measurements according to an exemplary embodiment. Thus, the algorithm presents an exemplary flowchart for stimulation determination based on state of sleep. This flowchart is exemplary and presents an exemplary method for some embodiments and it is noted that other embodiments may not necessarily follow this flowchart in whole or in part.

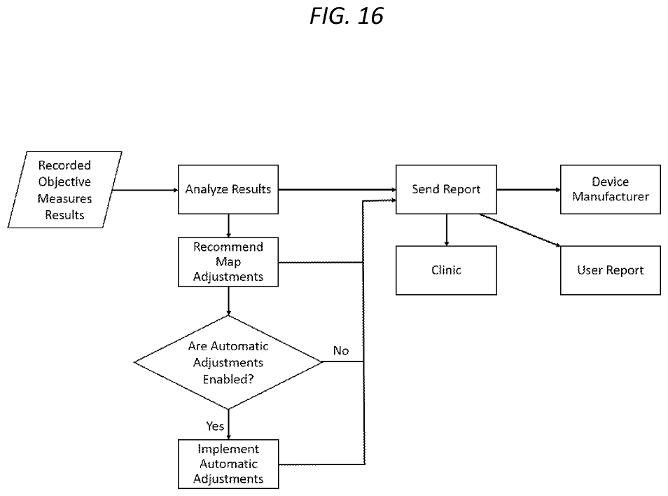

FIG. 16 presents another exemplary algorithm for an exemplary method that can be utilized in some embodiments. This algorithm is directed towards the distribution of data that is gathered during sleep.

It is noted that in at least some exemplary embodiments, some if not all of the stimulation that is executed in a given temporal period associated with recipient sleep occurs outside of stage I and/or stage II sleep. In an exemplary embodiment, on a temporal basis, for a given sleep period (e.g., an 8-hour period), temporally measured, no more than 30, 25, 20, 15, 10, 9, 8, 7, 6, 5, 4, 3, 2, or 1% of the stimulation is provided during stage I and/or stage II sleep. Embodiments include avoiding or otherwise not executing stimulation that is based in a sound that is relevant or emotionally charged the recipient. Accordingly, in an exemplary embodiment, pretesting is performed or pre-evaluation is performed that evaluates what sounds would be emotionally charged and/or relevant to the recipient, in order to avoid such during testing, which sound are avoided, and/or to identify sounds that are not relevant and/or not emotionally charged with respect to a given recipient, which sound utilized during the testing.

As will be noted below, in some instances, the testing/measurements can be based on what would be relatively large sounds. That is, in some embodiments, such as for example, EcoG testing, the sounds should be loud. In an exemplary embodiment, the methods associated with applying stimulation include gradually building or increasing the magnitude of a stimulation so that it is not shocking or otherwise startling to the recipient, and otherwise increases the likelihood that the recipient will sleep through the noise. Still further, in an exemplary embodiment, the stimulation is provided in a sound context of a common household sound. By way of example only and not by way of limitation, a household central air-conditioning system can include a blower that makes considerable noise when such is activated. People will typically sleep through the activation of such, even though the noise is relatively loud. In this regard, the people have been conditioned to that noise. Accordingly, an exemplary embodiment entails identifying noises to which the recipient has been conditioned to sleep through and utilizing those noises with respect to the application is stimulation to the recipient. It is noted that in an exemplary embodiment, those noises can be potentially increased in magnitude relative to the natural noise, if such still maintains the sleep of the recipient. In this regard, a type of noise can be identified that is common or otherwise experienced while the recipient is sleeping, which noise does not wake the recipient, and the magnitude that noise can be increased when applying the stimulation. Alternatively, and/or in addition to this, the common/condition noises as part of a method that reduces the difference between silence and the noise associated with stimulation. By way of example only and not by way of limitation, a noise that might otherwise wake a recipient in the absence of noise may not necessarily wake the recipient if there is other noise that is different than that noise. By way of example only and not by way of limitation, white noise is a type of noise that can be utilized to mask the difference between a peak sound, such as a door closing.

Accordingly, in an exemplary embodiment, there is the action of identifying common sounds that are present when the recipient is sleeping, and utilizing those sounds as part of the stimulation that is applied during implementation the method actions detailed herein.

Corollary to this is that in an exemplary embodiment, the recipient is slowly conditioned to the sounds associated with the testing. In an exemplary embodiment, the sound may not be a sound that normally exists during recipient night, but is created as such over a period of time, such as by first introducing the sound brings straight three and/or stage IV sleep at a low volume, and then gradually increasing the volume, and/or then expanding the location of the application of that sound to the stage I and/or stage II sleep. Upon sufficient conditioning, though sounds can be utilized as part of the methods detailed herein.

Some embodiments include polysomnogram application to evaluate the state of sleep. Exemplary embodiments can utilize brain waves and/or eye-movement and/or evaluation of heart rate, such as via ECG, muscle tension, oxygen levels, breathing and/or airflow, and/or the utilization of a microphone, the latter being utilized to record frequency and/or volume of snoring activity. Any one or more or all of these methods can be utilized singularly or collectively to evaluate or otherwise obtain data to determine a state of sleep of a recipient.

Embodiments also include collecting data from an accelerometer that senses movement. In an exemplary embodiment, the device measures how much movement the recipient makes during sleep, and this data is then used in an algorithm to estimate sleep time and/or quality. In some exemplary embodiments, there is a method that includes obtaining data regarding the movements of a given recipient as correlated to the state of sleep of that recipient, and building up a database over time, which databases then used to determine a state of sleep of that given recipient. Again, any feature can be utilized in combination, and thus, an exemplary embodiment includes utilizing a movement detector along with, for example, a heart rate monitor.

In an exemplary embodiment, there is a method of monitoring EEG utilizing electrodes that are not external electrodes and/or that are not implanted for the specific purpose of monitoring EEG. In an exemplary embodiment, there are no external electrodes. That said, in some alternate embodiments, external electrodes that are attached to the scalp and the temporary manner are utilized in at least some exemplary embodiments to obtain EEG measurements.

In an exemplary embodiment, again, as noted above, a microphone can be utilized to capture environmental sounds and/or the sounds of sleep, such as, for example, breathing patterns, snoring, rustling of sheets, sleep talk, etc. This microphone can be part of the prosthesis or can be a separate microphone entirely. As with the other examples herein, in an exemplary embodiment, a data acquisition can be executed prior to the measurements, where data is collected about the sounds the recipient makes an as correlated to a given state of sleep so that later the state of sleep can be determined based on the sounds.

While the embodiments detailed above have generally been focused on utilizing passive techniques to determine a state asleep, in an alternate embodiment, more active techniques can be utilized. By way of example only and not by way of limitation, a stimulus, such as annoyance stimulation or general sound stimulation, can be provided to estimate the wakefulness of the recipient or otherwise estimate the state of sleep of the recipient. By way of example only and not by way of limitation, if a sound having a decibel level in a certain level does not wake the recipient, it is possible to treat this is a latent variable and thus deduce that the recipient is in stage III and/or stage IV sleep if that sound at that decibel level woke the recipient during other states of sleep.

Moreover, it is noted that in at least some exemplary embodiments, a detailed sleep study of a given recipient can be made, or at least a professional or quasi-professional sleep study of the recipient can be executed. That is, when developing the baseline information, the recipient can be studied, and certain actions associated with the recipient that can be detected utilizing the techniques detailed herein can be correlated to a given state of sleep for that recipient, which then can be utilized to determine the status sleep when testing is implemented. Further, in an exemplary embodiment, the stimulation can be provided to the recipient that would wake the recipient or not wake the recipient's, which is correlated to a given state asleep, which thus can be utilized to build the database associated with the sleep patterns of the recipient, and thus the stimulation can be utilized to determine the status sleep that the recipient is an based on whether or not the recipient wakes.

It is briefly noted that in at least some exemplary embodiments, the stimuli that is utilized is very very low rate of stimulation. In an exemplary embodiment, the rate is 1 measure per half second, ¾ths of a second, 1 second, 2, 3, 4, 5, 6, 7, 8, 9, 10, 11, 12, 13, 14, or 15 seconds, etc.

An exemplary embodiment includes a method that includes automatically monitoring input indicative of a state of sleep of the recipient while the recipient is asleep, determining, based on the automatic monitoring, that at least one of an elimination of the recipient being asleep or a change in a state of the asleep has occurred. Further, concomitant with the teachings detailed herein, this method can include the action of automatically decreasing a magnitude of or eliminating entirely stimulation applied to the recipient during the action of implementing measurements upon the determination. Further, an exemplary embodiment includes automatically fitting or refitting the hearing prosthesis based at least in part on any of the method actions detailed herein. Concomitant with the automated fitting embodiments, embodiments can include code for automatically fitting or refitting the hearing prosthesis based at least in part on the measurement method.

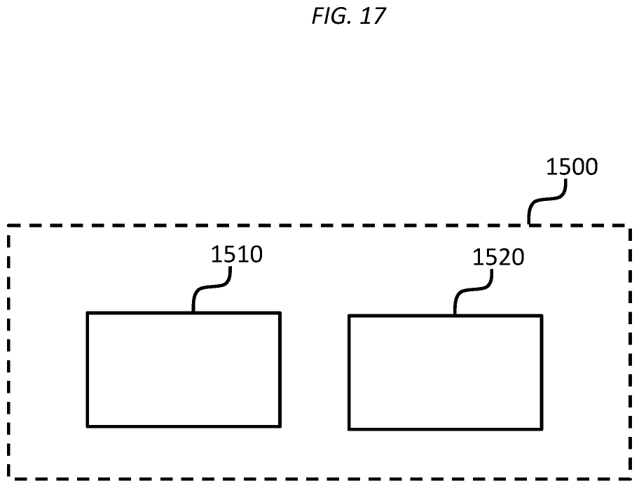

FIG. 17 presents a functional diagram of a system 1500 accordingly to an exemplary embodiment, including a first subsystem 1510 and a second subsystem 1520. The first subsystem, 1510, is configured to obtain data indicative of a sleep state of a recipient of a sensory prosthesis. In this regard, in an exemplary embodiment, the first subsystem can correspond to any of the teachings detailed herein or any other system that can enable obtaining data indicative of a sleep state of the recipient. Further, second subsystem 1520 is configured to execute measurements of the recipient. In an exemplary embodiment, the first subsystem or another subsystem can evaluate the obtained data indicative of a sleep state of the recipient, and determine that measurements taking should be executed, and thus direct the second subsystem to commence the execution of measurements or otherwise indicate to the second subsystem that testing can be commenced. In an exemplary embodiment, the subsystems can all be embodied in a single hearing prosthesis, while in other embodiments, one or both of the subsystems, such as for example, the first subsystem, is separate and distinct and otherwise not a part of the hearing prostheses.

It is noted that this system, system 1500, does not require the ability to analyze the obtained data. Instead, there is only the requirement that the system be able to obtain the data. In this regard, a prosthesis can be utilized in combination with a separate device, whether that be in the bedroom with the recipient were located remotely there from, to execute one or more the method actions detailed herein. Accordingly, in an exemplary embodiment, system 1500 can be utilized to obtain the data indicative of the sleep state of the recipient. This can be done by a prosthesis, such as a hearing prosthesis. This obtained data can then be provided to another device, separate from the hearing prostheses, such as a smart device for a device that is remote on the hearing prosthesis, such as a mainframe computer in signal communication directly or indirectly with the hearing prostheses via the Internet or the like, where that device analyzes the obtained data. Based on the analysis, the device can then provide input to the prosthesis to commence measurement taking. Thus, in an exemplary embodiment, system 1500 can correspond to the hearing prosthesis. Note also that in an exemplary embodiment, the first subsystem or another subsystem can also be configured to actually analyze the obtained data, thus executing the methods associated with the system 1500 utilizing an integrated device/utilizing only the prostheses. That said, again, subsystem 1510 can be separate from the prosthesis and/or subsystem 1520 can be separate from the prostheses. Moreover, in an exemplary embodiment, system 1500 can be completely separate from any prosthesis. Thus, in an exemplary embodiment, system 1500 is configured to analyze the sleep state of the recipient and/or implement a measurement regime from amongst a plurality of measurement regimes based on the analysis (more on measurement regimes below). The processor of the prostheses can be programmed or otherwise configured to execute the analysis utilizing any algorithm appropriate such as, for example, a lookup table where data that is received is compared to prestored or predetermined data, and based on a comparison between the two, and action is correlated to the comparison and the processor instructs componentry of the system to execute that action.

Still, in an exemplary embodiment, subsystem 1510 is part of a hearing prosthesis. Indeed, in an exemplary embodiment, the system is configured to utilize hearing prosthesis componentry to obtain the data that is obtained. By “hearing prostheses componentry,” it is meant components that are traditionally understood by the person of ordinary skill in the art to be readily expected to be present in the prostheses. For example, a microphone, an accelerometer, the electrodes of the cochlear implant that are utilized to evoke a hearing percept, are all “hearing prostheses componentry.” Conversely, separate electrodes that are not utilized to evoke a hearing percept, even in the case of a cochlear implant, would not be hearing prosthesis componentry. Instead, those would be extra componentry that are added to a hearing prosthesis. Further by way of example, devices that utilize eye-movement to determine the status sleep of a recipient would not be hearing prostheses componentry, although it is possible that such would be retinal implant prostheses componentry. A remote microphone that is not part of a hearing prostheses is also not hearing prostheses componentry, even though a microphone is almost always present in a hearing prosthesis. This is not to say that at some exemplary systems do not utilize non-hearing prosthesis componentry. Indeed, some exemplary embodiments specifically utilize eye tracking devices like to determine sleep state. Accordingly, an exemplary embodiment includes a system that is configured to utilize non-hearing prosthesis componentry to obtain the data that is obtained. In an exemplary embodiment, the system is configured to utilize both hearing prosthesis componentry and non-hearing prosthesis componentry to obtain the data.

As noted above, in at least some exemplary embodiments, the teachings detailed herein are executed without the recipient dedicating time to those measurements associated with the actions. This is not to say that the recipient does not have to set up or otherwise gauge the system for the testing and measurements. This is to say that when those measurements are taken, the recipient is not doing anything that the recipient would not normally do, irrespective of the testing, all other things being equal. Indeed, many of the actions detailed herein are executed while the recipient is sleeping. This as contrasted to an exemplary scenario where the recipient must affirmatively visit a clinician or otherwise affirmatively participate in testing. Again, in some embodiments, many if not most, if not all of the measurements are taken while the recipient is not conscious.

In embodiments where the system 1500 is a hearing prosthesis, in some exemplary embodiments of such, the system can be configured to have the hearing prosthesis evoke a hearing percept indicative of background noise, and the system is configured to interleave measurement stimulus in the background noise. Again, in an exemplary embodiment, a white noise can be applied to the prostheses while the recipient is sleeping or while the recipient is in pre-sleep, which white noise can include the stimulus for the measurements.

Concomitant with the teachings detailed above where the cochlear implant is utilized for EEG and/or EMG data collection, in an exemplary embodiment, system 1500 is cochlear implant including electrodes located in the cochlea and return electrodes located outside the cochlea. In this exemplary embodiment, the electrodes are part of the first sub-system and the system is configured to use the electrodes to monitor an EEG and/or EMG of the recipient to obtain the data indicative of the sleep state of the recipient. Still further, in an exemplary embodiment, the cochlear implant is configured to utilize those electrodes to evoke a hearing percept before and/or after and/or during the monitoring of the EEG and/or EMG.

Some embodiments of system 1500 include a system that is configured to automatically analyze the results of the measurements, identify changes to settings of the prosthesis and/or identify new settings of the prosthesis based on the analysis and/or automatically implement the change of the setting in the prosthesis or provide the new setting to the prosthesis. In an exemplary embodiment, this can enable the kinds of incremental and consistent or continuous updating that other systems cannot provide. Again, in an exemplary embodiment, adjustments can be made to the prosthesis one a daily or weekly basis without intervention by a healthcare professional and/or without having to participate in testing directed by or otherwise under the control of the health care professional.

The adjustments to the settings detailed herein/adjustments to the maps detailed herein can include, based on the measurements, any one or more or all of T and/or C adjustment for audibility daily or as needed, rate changes, assessment of neural health, changes to Focused Multipolar channels (channel weights and/or degree of focusing per channel, etc.), improved maps for infants, children, and/or other populations who do not have reliable behavioral responses.

Moreover, irrespective of the development of new or revised maps or adjustments, the teachings detailed herein can be utilized for the purpose of gathering of data immediately after activation, the tracking of changes over time and/or the conditioning to improve impedances, and/or the detection of “bad” channels and/or early indication of device failure. Indeed, it may not be the case that an adjustment is made to the prosthesis, such as the case where there is device failure, where the device will likely have to be explanted. In an exemplary embodiment, there is the action of conditioning to improve impedances upon wake-up. In any event, in at least some exemplary embodiments, the louder objective measures can be executed in a manner that wakes the person up or otherwise are correlated to a wake-up.

The following include exemplary and non/exhaustive measurements that can be made with respect to the measurement actions herein:
Impedances
Transimpedances
Electrocochleography (EcoG)
Electrically Evoked Compound Action Potential (ECAP)/Neural Response Telemetry (NRT)
Electrical Stapedial Reflex Threshold (ESRT)
Electrical Auditory Brainstem Response (EABR)
Electrical Auditory Steady State Response (EASSR)
P1-N1-P2 complex/Mismatched Negativity (MMN)
Binaural Interaction Component (BIC)
Channel Interactions
Cortical Auditory Evoked Potentials (CAEPs)

In an exemplary embodiment, impedance measurements are taken during stage I and/or stage II sleep and/or in any of the stages of sleep and/or during pre-sleep. In an exemplary embodiment, there is the action of taking a measure of the opposition to electrical current flow across an electrode. This can be considered an impedance measurement. In an exemplary embodiment, the level of stimulation is considered very soft and takes about 1 to 10 minutes or any temporal period there between, such as about five minutes. In an exemplary embodiment, this can be utilized to determine shortened electrodes and/or to identify open circuits. In an exemplary embodiment, upon a determination that there exists a shortened electrode and/or an open circuit, the channels associated therewith might be removed or otherwise weighted in a different manner than that which would be the case.

In an exemplary embodiment, transimpedance measurements are taken during stage I and/or stage II sleep and/or in any of the stages of sleep and/or during pre-sleep. In an exemplary embodiment, there is the action of applying current to one or more or all of the intracochlear electrode in a MP configuration and measuring corresponding voltage is measured at one or more all the other intracochlear electrodes. A trans-impedance matrix made up of the ratio of the voltage to the current can be generated, which represents the current spread functions for the stimulating electrode array. In an exemplary embodiment, the level of stimulation is considered very soft and takes about 1 to 10 minutes or any temporal period there between, such as about five minutes. In an exemplary embodiment, this can be used to create weights for focused multipolar stimulation and/or to help determine the presence of a tip fold over for electrode array placement post-surgery. Such can provide information to an audiologist or the like that will enable him or her to adjust a map or the like or otherwise make an adjustment to the cochlear implant settings.

In an exemplary embodiment, EcoG measurements are taken during pre-sleep and/or wake-up, and the stimulation associated there with can correspond to the alarm that wakes the recipient. That said, in an exemplary embodiment, depending on the recipient, these measurements can be executed during stage III and/or stage IV sleep. In some embodiments, the stimulation associated with these measurements is combined or otherwise utilized as background noise or white noise or a noise that is not relevant to the recipient, where the noise might otherwise wake the recipient. In an exemplary embodiment, there is the action of recording of the electrical potentials of the cochlea. EcoG measurements can involve measurement of the stimulus-related cochlear potentials (as opposed to the resting potentials), and often includes measurement of the whole nerve or compound action potential (AP) of the auditory nerve. In some embodiments, this can include measurements of the cochlear microphonic (CM), cochlear summating potential (SP), and AP measured independently or in various combinations. In an exemplary embodiment, this can be executed utilizing loud stimulus. EcoG can often take about 30 minutes.

In an exemplary embodiment, the EcoG measurements are utilized to diagnose and/or assess and/or monitor Meniere's disease/endolymphatic hydrops. In an exemplary embodiment, these measurements are utilized to enhance or otherwise determine how to enhance wave I of the ABR in the presence of hearing loss or when less than optimal recording conditions were used to obtain wave I. Further, in an exemplary embodiment, the measurements can be utilized to measure and/or monitor the cochlear and auditory nerve function during surgery involving the auditory periphery and/or diagnose auditory neuropathy spectrum disorder (ANSD).

In an exemplary embodiment, Electrically Evoked Compound Action Potential (ECAP)/Neural Response Telemetry (NRT) measurements are taken during pre-sleep and/or wake-up, and the stimulation associated there with can correspond to the alarm that wakes the recipient. That said, in an exemplary embodiment, depending on the recipient, these measurements can be executed during stage III and/or stage IV sleep. In an exemplary embodiment, ECAP represents a synchronous response from electrically stimulated auditory nerve fibers. Neural Response Telemetry (NRT) is the ECAP telemetry software used in Custom Sound (AutoNRT) and Custom Sound EP. These can be typically applied with a loud stimulus. AutoNRT can often take between 1 to 10 minutes or a value therebetween, such as 5 minutes. The time for other ECAP/NRT testing depends on the test parameters and how much testing needs to be completed.

Exemplary embodiments include utilizing ECAP measures to guide mapping and/or for assistance in programming the speech processor for individuals who cannot provide reliable behavioral responses. These measures can also be used for verification or confirmation of the accuracy of questionable behavioral responses. These measures can also be used for objective verification of auditory nerve function in response to electrical stimulation and/or objective verification of electrode/device function in surgery and post-surgery. These measures can also be used to determine or otherwise identify amplitude growth functions and spread of excitation in the cochlea.

In an exemplary embodiment, Electrical Stapedial Reflex Threshold (ESRT) measurements are taken during pre-sleep and/or wake-up, and the stimulation associated there with can correspond to the alarm that wakes the recipient. That said, in an exemplary embodiment, depending on the recipient, these measurements can be executed during stage III and/or stage IV sleep. This can entail electrically elicited middle-ear muscle reflexes monitoring. In an exemplary embodiment, the stimulation that is utilized for these measurements is loud, and the testing can entail testing for tens of minutes, including about a half hour or so.

In an exemplary embodiment, the measures are analyzed to determine conformance with respect to the responsiveness to electrical stimulation, to guide initial programming and/or to create maps, to monitor the recipient's over time, and/or to program hearing prostheses that will be used for multiple-handicap children and/or difficult to condition children and/or adults with long-duration of deafness.

In an exemplary embodiment, Electrical Auditory Brainstem Response (EABR) measurements are taken during pre-sleep and/or wake-up, and the stimulation associated there with can correspond to the alarm that wakes the recipient. That said, in an exemplary embodiment, depending on the recipient, these measurements can be executed during stage III and/or stage IV sleep. In an exemplary embodiment, measurements are taken of auditory brainstem response (ABR) with regard to neural synchrony along the auditory pathway through the brainstem. ABR can be performed by electrical stimulation through the cochlear implant (EABR). In an exemplary embodiment, the stimulation that is utilized for these measurements is loud. There can be utilitarian value with respect to analyzing the measurements these of the execution of a functional evaluation of the auditory system between the time of initial implant activation and after chronic cochlear implant use.

In an exemplary embodiment, Electrical Auditory Steady State Response (EASSR) measurements are taken during pre-sleep and/or wake-up, and the stimulation associated there with can correspond to the alarm that wakes the recipient. That said, in an exemplary embodiment, depending on the recipient, these measurements can be executed during stage III and/or stage IV sleep. In an exemplary embodiment, measurements are taken with respect to neural responses to periodic electrical stimulation. The stimulation that is applied is loud, and the utilitarian value with respect to analyzing the measurements can include predicting the threshold levels and/or providing objective measurements of site-specific temporal sensitivity.

Still further, in an exemplary embodiment, the acoustic change complex (ACC) measurements are taken. In at least some exemplary embodiments, when obtained in response to an acoustic change within an ongoing sound, the resulting waveform is referred to as the ACC. When elicited, the ACC indicates that the brain has detected changes within a sound and the patient has the neural capacity to discriminate the sounds. In fact, results of several studies have shown that the ACC amplitude increases with increasing magnitude of acoustic changes in intensity, spectrum, and gap duration. In addition, the ACC can be reliably recorded with good test-retest reliability not only from listeners with normal hearing but also from individuals with hearing loss, hearing aids, and cochlear implants. The ACC can be obtained even in the absence of attention, and requires relatively few stimulus presentations to record a response with a good signal-to-noise ratio. In an exemplary embodiment, the measurements can have utilitarian value with respect to identifying reasonable agreement with behavioral measures. ACC thus can be utilized for the objective clinical evaluation of auditory discrimination and/or speech perception capacity.

Moreover, measurements for P1-N1-P2 complex/Mismatched Negativity (MMN) can be implemented. In some embodiments, the P1-N1-P2 response is an obligatory cortical AEP, passive recording of this response that is done. This response is typically always present in a healthy auditory system when subjects are awake (with some differences in morphology in children). It can be elicited by the onset of a sound such as a click or a tone, or it can be elicited by a change in a stimulus. The other two cortical AEPs, the MMN and the P300, are obtained with oddball paradigm presentations: where a standard stimulus is presented most of the time and a deviant stimulus is presented occasionally (usually 10-20% of the time). The MMN can be recorded in passive listening conditions; this response is automatic, but it is not always present. The other potential, the P300 is also elicited using an oddball paradigm but in this case the recording is not passive; subjects' participation is required (typically clients are asked to count the deviants). In at least to some exemplary embodiments, the measurements can have utilitarian value with respect to ascertaining the P1-N1-P2 recorded from the auditory cortex following presentation of an acoustic stimulus, which can be utilized to identify or otherwise ascertain an understanding of the neural encoding of a sound signal.

In an exemplary embodiment, the measurements are Binaural Interaction Component (BIC) measurements. In an exemplary embodiment, these measurements are limited to bilateral cochlear implant recipients. In an exemplary embodiment, the binaural interaction component (BIC) is obtained by subtracting the summed auditory brainstem response (ABR) in the monaural stimulus mode from the ABR obtained in the binaural stimulus mode. In an exemplary embodiment, these measurements once analyzed provide for objective measure of binaural interaction, possible diagnostic tools in CAPD patients, the determination of pitch mismatch of electrode place between ears and/or the indirect assessment of localization and sound segregation.

Also, as noted above, in an exemplary embodiment, the measurements are utilized to ascertain or otherwise evaluate features associated with channel interaction if such exists in the first instance. In at least some exemplary embodiments, channel interactions are measured between neighboring probe and perturber channels. Embodiments include analyzing the measurements and adjusting the focus of the channels so as to minimize and/or eliminate the interaction. In an exemplary embodiment, the adjustment is an iterative and/or ongoing process that is made in tiny steps or iterative steps. Accordingly, the teachings detailed herein can enable channel adjustment/channel interaction evaluation in a recipient efficient manner in that the recipient need not be involved or otherwise spend time with respect to the testing. There is utilitarian value with respect to evaluating channel interactions in that in some embodiments, such can determine the maximum level of focusing. When the minimum interaction point for each channel is reached, the focusing is at the optimal level in at least some exemplary embodiments. Increased focusing from the optimal level would introduce more channel interactions. In this regard, in an exemplary embodiment, the measurements herein can be applicable to identifying channel interactions of the amount of channel interactions. In an exemplary embodiment, the measurements are analyzed to determine how the channel should be adjusted. Adjustments are made and then stimulation is again provided along with the accompanying measurements, and then the measurements are analyzed, and the process is repeated until the optimal level is determined.

It is noted that the channel interaction analysis can potentially be executed a number of times during a given sleep. Moreover, the settings can be repeatedly adjusted during a given sleep.

In an exemplary embodiment, the level of stimulation is soft. In an exemplary embodiment, measurements for channel interaction determination are executed during the pre-sleep period, stage I, II, III and/or IV sleep.

In view of the above, it can be seen that in at least some exemplary embodiments, there is a system where the measurements include at least one of impedance measurements, transimpedance measures, ECoG, ECAP, NRT, ESRT, EABR, EASSR, MMN, BIC, channel interaction measurements, or ECAEPs and the system is configured to execute measurements while the recipient is asleep without waking the recipient.

It is noted that while many of the teachings detailed herein are directed towards applying stimulation for the purposes of measurement, and why the following is not mutually exclusive there with, some embodiments also include the action of applying stimulation during the temporal periods detailed herein in the manner detailed herein as part of an auditory training method. By way of example only and not by way of limitation, there can be the action of presenting words and/or phonemes during sleep. In an exemplary embodiment, there can also be an analysis of problem phonemes prior to a given sleep period, and the presentation during the sleep period is such that the associated simulation corresponds to a tailored auditory training program for that recipient.

Accordingly, in an exemplary embodiment, the actions of automatically controlling the hearing prostheses to provide the stimulation based on a determination of the sleep state of the recipient can correspond to providing stimulation for auditory training purposes.

In an exemplary embodiment, the auditory training takes place during stage I and/or stage II of sleep. That said, in some alternate embodiments, it can take place during the later stages of sleep.

As noted above, some of the method actions detailed herein are implemented by a hearing prosthesis, while in other embodiments, some of the method actions are implemented by a device that is not a hearing prosthesis, while in other embodiments, a given method action can be executed by the prostheses and/or another device that is not a hearing prosthesis. Accordingly, any method action herein that is disclosed with respect to a hearing prosthesis corresponds to a disclosure of the execution of that method action by something that is not a hearing prosthesis, such as for example, a smart phone or a smart device or a personal computer or a mainframe computer, etc. Also, any disclosure herein of a method action that is executed by something that is not a hearing prosthesis corresponds to a disclosure of a method action that is executed by a hearing prosthesis. Any disclosure of a method action executed by one device constitutes a disclosure of a method action executed by any of the other devices herein. Note all of this is subject to the proviso that such is not otherwise indicated and/or that the art enables such.

Embodiments include a general-purpose microprocessor or a general-purpose computer that is programmed and configured to execute one or more of the method actions detailed herein. In an exemplary embodiment, a processor of a hearing prosthesis is programmed and/or configured to execute one or more the method actions detailed herein.

It is noted that while the teachings detailed herein are described in terms of an electrical stimulating device in the form of a cochlear implant, it is noted that alternate embodiments are applicable to other types of stimulating devices. By way of example only and not by way of limitation, the teachings detailed herein and/or variations thereof can be applicable to a bone conduction device, a Direct Acoustic Cochlear Implant, or traditional hearing aids, at least those having channel features.

As noted above, at least some of the method actions can be executed at a location remote from where another method action is located. For example, it is noted that an exemplary embodiment entails executing some or all of the method actions detailed herein, where the recipient of the hearing prosthesis is located remotely (e.g., geographically distant) from where at least some of the method actions detailed herein are executed (e.g., any method action detailed herein that can be executed by, for example, a computer or other processor located at a remote location). For example, any of the methods detailed herein could be executed via internet communication with the hearing prosthesis and the user interface 314 and/or the hearing implant fitting system 306 (e.g., communication link 308 of FIG. 3 can be an internet connection or a wired or wireless connection). Still further by example, with respect to a given method, one or more method actions can be executed at one location (controlled by the audiologist 304 at another location geographically remote from the one location), and one or more other method actions can be executed at the location where the audiologist 304 is located. That is, any method action herein can be executed at one location, and any method action herein can be executed at another location, and so on, providing that the teachings detailed herein and/or variations thereof can be practiced.

It is further noted that in an alternate embodiment, one or more of the method actions detailed herein are executed by the recipient of the cochlear implant. Indeed, in an exemplary embodiment, there is a system that enables a recipient to execute, in conjunction with the system, the method actions detailed herein such that the cochlear implant can be remapped without any additional input from a clinician or the like.

It is noted that any disclosure of a method action detailed herein corresponds to a disclosure of a corresponding system and/or device for executing that method action, in at least some embodiments, automatically. It is further noted that any disclosure of an apparatus or system herein corresponds to a disclosure of a method of operating that apparatus. It is also noted that any disclosure of any method action detailed herein further includes a disclosure of executing that method action in an automated fashion, as well as a device for executing those method actions in the automated manner.

It is further noted that any disclosure of a fitting method herein corresponds to a hearing prosthesis or hearing device fitted according to that method.

Any disclosure herein of any method action of making a device and/or establishing a system corresponds to a device and/or system that results from that method action. Any disclosure herein of any device and/or system corresponds to a disclosure of a method of making that device and/or system and/or otherwise establishing that device and/or system.

Any embodiment or any feature disclosed herein can be utilized in combination with any one or more of any other embodiment or any feature disclosed herein, unless otherwise noted and/or unless the art does not enable such. Any embodiment or any feature disclosed herein can be explicitly excluded from combination with any one or more of any other embodiment or any feature disclosed herein, unless otherwise noted and/or unless the art does not enable such. Thus, any disclosure herein of any given feature embodiment corresponds to a disclosure of a device and/or system and/or method that specifically does not have that given feature and/or embodiment.

While various embodiments of the present invention have been described above, it should be understood that they have been presented by way of example only, and not limitation. It will be apparent to persons skilled in the relevant art that various changes in form and detail can be made therein without departing from the scope of the invention.

1. A method, comprising:
providing stimulation to a recipient of a hearing prosthesis, wherein
the stimulation is provided at temporal locations associated with sleep of the recipient, and
the stimulation is at least one of part of a measurement method or an auditory training method.

2. The method of claim 1, wherein:
the stimulation is part of an auditory training method; and
the temporal locations are temporal locations corresponding to the recipient being asleep.

3. The method of claim 1, wherein:
the stimulation is part of a measurement method executed at the temporal locations;
the action of providing the stimulation executes measurements of the recipient; and
the temporal locations are temporal locations corresponding to at least one of the recipient in a going to sleep state, the recipient asleep or the recipient just waking up.

4. The method of claim 3, wherein:
the stimulation is audible; and
the temporal location corresponds to the recipient being asleep.

5. The method of claim 3, further comprising:
automatically fitting or refitting the hearing prosthesis based at least in part on the measurement method.

6. The method of claim 1, wherein:
the stimulation is part of a measurement method executed at the temporal locations;
the stimulation is provided at an audible level and measurements are taken based on the stimulation while the recipient is in a going to sleep state and a sleep state; and
the hearing prosthesis is a cochlear implant.

7. The method of claim 1, further comprising:
automatically determining a sleep state of the recipient using the hearing prosthesis; and
automatically controlling the hearing prosthesis to provide the stimulation based on the determination.

8-10. (canceled)

11. A method, comprising:
receiving input indicative of measurements executed using a hearing prosthesis while the recipient thereof is sleeping;
analyzing the received input; and
at least one of adjusting a setting of the hearing prosthesis or loading a new setting of the hearing prosthesis based on the analysis.

12. The method of claim 11, wherein:
the action of adjusting or loading is executed automatically based on the analysis.

13. The method of claim 11, wherein:
the prosthesis is a cochlear implant; and
the action of adjusting or loading causes the cochlear implant to stimulate the recipient consistently in a different manner for a given input than that which would have been the case in the absence of the adjusting or loading, all other things being equal.

14. (canceled)

15. The method of claim 11, wherein:
the actions of adjusting or loading is executed automatically in real time with the execution of the measurements.

16. (canceled)

17. The method of claim 11, wherein:
the action of receiving input indicative of measurements executed using a hearing prosthesis while the recipient thereof is sleeping is executed via electronic communication to a location remote from where the recipient was sleeping; and
the analysis and the actions of adjusting and loading are executed with the assistance of a healthcare professional remote from the recipient.

18. The method of claim 11, wherein:
the hearing prosthesis includes a plurality of channels; and
the method further includes:
mapping parameters on a per channel basis based on the analyzed received input, wherein the action of adjusting or loading results in a change to a channel of the hearing prosthesis and no change to another channel of the hearing prosthesis.

19. The method of claim 11, further comprising:
performing measurements that form the basis of the input indicative of the measurements, the measurements being executed in part by providing stimulus to the recipient, wherein the stimulus is embedded in a sound regime that is associated with sleep of the recipient.

20. A non-transitory computer readable medium having recorded thereon, a computer program for executing a method, the program including:
code for determining a feature indicative of a sleep state of a recipient of a hearing prosthesis; and
code for implementing measurements of the recipient based on the determination of the sleep state.

21. The medium of claim 20, further comprising:
code for analyzing input indicative of the sleep state of the recipient, wherein
the code for determining a feature indicative of a sleep state is code for determining the sleep state of the recipient that uses the analysis of the input indicative of the sleep state of the recipient.

22. The medium of claim 20, further comprising:
code for analyzing a first input indicative that the recipient is in a first sleep state, wherein the code for determining a feature indicative of a sleep state is code for determining the sleep state of the recipient that includes code for determining that the recipient is in the first sleep state based on the analysis of the first input; and
code for, based on the determination that the recipient is in the first sleep state, automatically implementing a first objective measurement regime from amongst a plurality of measurement regimes, thus executing the action of implementing the measures of the recipient.

23. The medium of claim 22, further comprising:
code for analyzing a second input indicative that the recipient is in a second sleep state, wherein the code for determining the sleep state of the recipient includes code for determining that the recipient is in the second sleep state based on the analysis of the second input; and
code for, based on the determination that the recipient is in the second sleep state, automatically implementing a second objective measurement regime from amongst the plurality of measurement regimes, thus executing the action of implementing the measures of the recipient, wherein the second objective measurement regime is different from the first objective measurement regime, and the second sleep state is different from the first sleep state.

24. The medium of claim 23, further comprising:
code for analyzing a third input indicative that the recipient is in a third sleep state, wherein the code for determining the sleep state of the recipient includes code for determining that the recipient is in the third sleep state based on the analysis of the third input; and
code for, based on the determination that the recipient is in the third sleep state, automatically implementing a third objective measurement regime from amongst the plurality of measurement regimes, thus executing the action of implementing the measures of the recipient, wherein the third objective measurement regime is different from the first objective measurement regime and the second objective measurement regime, and the third sleep state is different from the second sleep state.

25. The medium of claim 20, further comprising:
code for automatically monitoring input indicative of a state of sleep of the recipient while the recipient is asleep;
code for determining, based on the automatic monitoring, that at least one of an elimination of the recipient being asleep or a change in a state of the asleep has occurred; and
code for automatically decreasing a magnitude of or eliminating entirely stimulation applied to the recipient during the action of implementing measurements upon the determination.

26-35. (canceled)
